(19) United States
(12) Patent Application Publication
(10) Pub. No.: US 2019/0076324 A1
(43) Pub. Date: Mar. 14, 2019

(54) DEVICES AND SYSTEMS WITH AN EXTERNAL DISPLACEMENT MECHANISM FOR CONTAMINANT-FREE ENGAGEMENT OF PHARMACEUTICAL VESSELS AND PHARMACEUTICAL ADMINISTRATION DEVICES

(71) Applicant: Mikael Naygauz, Forrest Hills, NY (US)

(72) Inventor: Mikael Naygauz, Forrest Hills, NY (US)

(21) Appl. No.: 16/100,654

(22) Filed: Aug. 10, 2018

Related U.S. Application Data
(60) Provisional application No. 62/619,795, filed on Jan. 20, 2018.
(60) Provisional application No. 62/619,920, filed on Jan. 22, 2018.
(60) Provisional application No. 62/562,490, filed on Sep. 25, 2017.
(60) Provisional application No. 62/564,933, filed on Sep. 28, 2017.
(60) Provisional application No. 62/612,267, filed on Dec. 29, 2017.
(60) Provisional application No. 62/566,414, filed on Sep. 30, 2017.
(60) Provisional application No. 62/582,922, filed on Nov. 7, 2017.
(60) Provisional application No. 62/560,195, filed on Sep. 19, 2017.
(60) Provisional application No. 62/563,072, filed on Sep. 26, 2017.
(60) Provisional application No. 62/558,502, filed on Sep. 14, 2017.
(60) Provisional application No. 62/651,000, filed on Mar. 30, 2018.
(60) Provisional application No. 62/644,185, filed on Mar. 16, 2018.
(60) Provisional application No. 62/625,334, filed on Feb. 1, 2018.
(60) Provisional application No. 62/653,356, filed on Apr. 5, 2018.
(60) Provisional application No. 62/657,809, filed on Apr. 15, 2018.
(60) Provisional application No. 62/662,743, filed on Apr. 25, 2018.
(60) Provisional application No. 62/669,948, filed on May 10, 2018.
(60) Provisional application No. 62/675,058, filed on May 22, 2018.

(51) Int. Cl. A61J 1/14; A61M 39/16; A61M 39/04; B65D 51/00

(52) U.S. Cl. CPC A61J 1/1443; A61J 1/1406; A61M 39/16; A61M 2039/1072; B65D 51/002; A61J 1/1418; A61M 39/04

(57) ABSTRACT

The present invention relates, in some embodiments thereof, to systems, devices and methods allowing for an engagement of two or more vessels or devices in a decontaminated manner. In some embodiments of the invention, the systems and devices of the invention include a first connection interface attached to a first vessel and a second connection interface attached to a second vessel, wherein the first connection interface and the second connection interface are configured to allow for an engagement between the first vessel and the second vessel, and wherein the first and second connection interfaces are further configured to externally displace from the engagement between the first vessel and the second vessel while a hermetically sealed connection is maintained between the first vessel and the second vessel.

### CROSS REFERENCE TO RELATED APPLICATIONS

This application claims the benefit of U.S. Provisional Patent Application No. 62/545,152 filed on Aug. 14, 2017; 62/558,502 filed on Sep. 14, 2017; 62/560,195 filed on Sep. 19, 2017; 62/562,490 filed on Sep. 25, 2017; 62/563,072 filed on Sep. 26, 2017; 62/564,933 filed on Sep. 28, 2017; 62/566,414 filed on Sep. 30, 2017; 62/582,922 filed on Nov. 7, 2017; 62/612,267 filed on Dec. 29, 2017; 62/619,795 filed Jan. 20, 2018; 62/619,920 filed Jan. 22, 2018; 62/625,334 filed on Feb. 1, 2018; 62/626,686 filed on Feb. 6, 2018; 62/631,729 filed on Feb. 17, 2018; 62/644,185 filed on Mar. 16, 2018; 62/649,483 filed on Mar. 28, 2018; 62/651,000 filed on Mar. 30, 2018; 62/651,149 filed on Mar. 31, 2018; 62/653,356 filed on Apr. 5, 2018; 62/657,806 filed on Apr. 14, 2018; 62/657,808 filed on Apr. 15, 2018; 62/657,809 filed on Apr. 15, 2018; 62/660,885 filed on Apr. 20, 2018; 62/662,721 filed on Apr. 25, 2018; 62/662,743 filed on Apr. 25, 2018; 62/664,933 filed on Apr. 30, 2018; 62/666,866 filed on May 4, 2018; 62/667,593 filed on May 6, 2018; 62/669,948 filed on May 10, 2018; 62/670,833 filed on May 13, 2018; 62/675,058 filed on May 22, 2018; 62/676,822 filed on May 25, 2018; 62/679,817 filed on Jun. 3, 2018; 62/680,576 filed on Jun. 4, 2018; 62/680,974 filed on Jun. 5, 2018; 62/681,884 filed on Jun. 7, 2018; 62/686,602 filed on Jun. 18, 2018; 62/686,612 filed on Jun. 18, 2018; and 62/690,260 filed on Jun. 26, 2018. The contents of the above applications are all incorporated by reference as if fully set forth herein in their entirety.

### FIELD OF THE INVENTION

The present invention relates, in some embodiments thereof, to systems, devices, and methods allowing for an engagement of two or more pharmaceutical vessels or pharmaceutical administration devices in a decontaminated manner. In some embodiments of the invention, the systems, devices and methods of the invention include a first connection interface attached to a first vessel (or administration device); and a second connection interface attached to a second vessel (or administration device), wherein the first connection interface and the second connection interface are configured to allow for an engagement between the first vessel and the second vessel, and wherein the first and second connection interfaces are further configured to externally displace from the engagement between the first vessel and the second vessel while a hermetically sealed connection is maintained between the first vessel and the second vessel.

### BACKGROUND OF THE INVENTION

The medicinal practice routinely involves administration of medical substances, such as, medicaments, fluids, nutritional substances and the alike, to patients or animals. The preparation and/or administration of such medicinal substances typically involves one or more transfers of those substances between pharmaceutical vessels or administration devices (such as, vials, syringes, infusion lines, connectors, etc.). Each such act of transferring substances between vessels or devices exposes the connection interfaces of the vessels and accordingly the medical substances themselves to contaminants present in ambient air or ambient air particles (e.g., bacteria, viruses, funguses, spores, pyrogens, dirt). In addition, connection interfaces are further prone to contaminations due to physical contact of the interfaces, for example, with nonsterile gloves, or devices.

Such contaminations are a major problem in the healthcare setting since contaminants, once invading within medicinal substances, may pose substantial danger if administered intracorporeally to patients.

Typical connection interfaces of pharmaceutical vessels or pharmaceutical administration devices include rubber bungs and/or stoppers covered by a cap and/or seal that can be flicked off and/or are removed prior to usage thereof. These rubber bungs/stoppers are used to allow penetration by a needle attached to a syringe or by other medical connectors. When the cap and/or seal is flicked off and/or removed, the rubber bung and/or stopper is exposed to ambient air and to contaminants present therein. Accordingly, exposure of connection interfaces to ambient air may involve contamination of the interfaces and consequently contamination of a beneficial substance to be provided to a patient.

Existing systems include U.S. Pat. Nos. D720,067; D717,947; D703,812; D690,418; D639,939; D637,713; 9,790,011; 9,775,777; 9,561,326; 9,493,281; 9,492,353; 9,309,020; 9,173,816; 9,168,203; 9,162,803; 9,039,672; 8,926,583; 8,827,978; 8,790,330; 8,662,985; 8,657,803; 8,622,985; 8,562,583; 8,545,475; 8,523,838; 8,491,563; 8,480,646; 8,449,521; 8,381,776; 8,336,587; 8,328,772; 8,287,513; 8,225,826; 8,075,550; 8,029,747; 7,998,134; 7,975,733; 7,942,860; 7,867,215; 7,744,581; 7,731,678; 7,387,216; 7,306,584; 6,875,203; 6,729,370; 6,715,520; 6,602,239; 6,409,708; 6,343,629; 6,162,199; 6,113,583; 6,063,068; 5,893,397; 5,876,380; 5,832,971; 5,807,374; 5,746,733; 5,569,235; 5,462,535; 5,405,326; 5,292,318; 5,279,582; 4,944,723; 4,932,947; 4,932,937; 4,919,657; 4,915,701; 4,826,489; 4,673,404; 4,564,054; 3,610,241; 3,605,743; 3,587,575; 3,583,399; 3,578,037; 3,556,099; 3,552,387; 3,406,686; 3,380,450; 3,375,825; 3,342,180; 3,330,282; 3,330,281; 3,306,290; 3,255,752; 3,253,592; 3,076,456; 2,972,991; 2,922,419; US20160262982; US20160038373; US20150209568; US20140183196; US20140016570; US20140007973; US20140000754; US20130184672; US20130006200; US20120209238; US20120209218; US20120203194; US20110284561; US20110186177; US20110125128; US20110108158; US20110098647; US20100249745; US20100198182; US20100152669; US20100147402; US20100036319; US20100004602; US20090057258; US20080312634; US20080223484; US20080171981; US20060276759; US20050215976; US20030199847; US20030187420; US20020130100; US20020115981; US20020099354; ES2577377T3; EP2852367B1; EP2666513; EP2155141B1.

In order to overcome this obstacle, the current medical practice involves swabbing the surface of a connection interface with a disinfecting agent, such as 70% isopropyl alcohol, prior to accessing the connection interface. Other methods include i.v. (intravenous) rooms which are used for the sterile preparation of i.v. medications. Such rooms, to keep medicinal preparations as sterile as possible, are equipped with special instruments including, hoods with air filtration systems (e.g., HEPA filters), ventilation systems and air pressure systems. Additionally, those rooms necessitate that the medical staff working in these rooms are properly garmented, are properly trained, and require aseptic techniques, and employ quality control and validation processes. These systems require regular upkeep by certified personnel and require regular cleaning. These systems are therefore expensive, labor intensive, and require regular maintenance and testing to assure that they are operating effectively. The above described systems and methods are either cumbersome and expensive or inefficient in addressing the problem of reducing/eliminating contaminants on connection interfaces.

Thus, there is a long felt and unmet need for pharmaceutical vessels, devices, systems and/or methods that afford transfer of medical substances in a sterile manner. There is a need for reliable, user friendly and cost-effective solutions allowing contaminant-free engagement of vessels and devices for drug preparation and administration processes.

### SUMMARY OF THE INVENTION

Objects of the invention are achieved by providing systems, devices, vessels and methods for engaging medical devices, such as vessels, in a decontaminated manner.

Objects of the invention are achieved by providing systems, devices, vessels and methods which are directed to transferring medical substances in an efficient, user-friendly and sterile manner.

The present invention is based on two or more connection interfaces, each of which is attached to a vessel. The connection interfaces are configured to connect between two or more medical vessels and while doing so, isolate and entrap therebetween ambient air particles. Such devices and systems allow for a hermetic and sterile connection between the vessels.

In an aspect of the invention, the present invention provides a decontamination system for the engagement of vessels, the system comprising: a first connection interface attached to a first vessel; and a second connection interface attached to a second vessel, wherein the first connection interface and the second connection interface are configured to allow for an engagement between the first vessel and the second vessel, and wherein the first connection interface and the second connection interface are further configured to externally displace from the engagement between the first vessel and the second vessel while a hermetically sealed connection is maintained between the first vessel and the second vessel.

In one or more embodiments, at least one of the first connection interface and the second connection interface is a film. In one or more embodiments, the film is a plastic or a metal alloy.

In one or more embodiments, upon the engagement between the first vessel and the second vessel, the first connection interface and the second connection interface are configured to slide with respect to the first vessel and the second vessel, thereby removing contaminants between the first vessel and the second vessel.

In one or more embodiments, the first connection interface is configured to seal and/or cover a port of the first vessel and/or a port of the second vessel.

In one or more embodiments, the first connection interface and the second connection interface are configured to seal and/or cover an aperture of the first vessel and an aperture of the second vessel, respectively.

In one or more embodiments, the decontamination system results in air particle-free engagement of the first vessel and the second vessel, wherein air particles that contact the first and second interfaces are entrapped by the first connection interface and the second connection interface.

In one or more embodiments, the system further comprises an engagement mechanism configured to provide a secure engagement between the first vessel and the second vessel. In one or more embodiments, the engagement mechanism is located on the first vessel and/or on the second vessel. In one or more embodiments, the engagement mechanism is selected from a thread, a luer, an adhesive, and a ratchet teeth mechanism. Optionally, a twisting, turning, and/or snapping motion is associated with the engagement. In an exemplary embodiment, a twisting motion is associated with a thread-luer mechanism. In an exemplary embodiment, a snapping motion is associated with a ratchet teeth mechanism. In one or more embodiments, when the first vessel and the second vessel engage via the engagement mechanism, an airtight seal is formed between the first vessel and the second vessel. In one or more embodiments, when the first vessel and the second vessel engage via the engagement mechanism a sealed aperture is formed between the first vessel and the second vessel.

In one or more embodiments, the sealed aperture formed between the first vessel and the second vessel allows for the displacement of at least one of the connection interfaces of the first vessel and the second vessel. In one or more embodiments, the sealed aperture is configured to displace at least one connection interface through the sealed aperture while maintaining the hermetic seal between the first vessel and the second vessel. In one or more embodiments, the system further comprises an engagement mechanism configured to provide a secure engagement between the first connection interface and the second connection interface. In one or more embodiments, the engagement mechanism is located on the first connection interface and/or on the second connection interface. In one or more embodiments, the engagement mechanism is selected from a thread, a luer, an adhesive, and a ratchet teeth mechanism. In one or more embodiments, the engagement mechanism includes a mechanical object that connects the first connection interface with the second connection interface providing for a simultaneous external displacement of the first and second connection interfaces. In one or more embodiments, the mechanical object is a protrusion, a dent, or a hook. In one or more embodiments, the engagement mechanism that secures the first connection interface to the second connection interface is an adhesive. In one or more embodiments, the engagement mechanism secures the first connection interface to the second connection interface forming an airtight seal between the first connection interface and the second connection interface. In one or more embodiments, the engagement mechanism is a thread on the first vessel configured to engage a complementary thread on the second vessel. In one or more embodiments, the engagement mechanism is a thread on the first connection interface configured to engage a complementary thread on the second connection interface. In one or more embodiments, the thread of at least one of the thread and complementary thread is a luer.

In one or more embodiments, the system further comprises a first rail mechanism on the first vessel configured to provide external displacement of the first connection interface. In one or more embodiments, the system further comprises a second rail mechanism on the second vessel configured to provide external displacement of the second connection interface.

In one or more embodiments, the system further comprises a first rail mechanism on the first vessel configured to provide external displacement of the first connection interface and configured to provide external displacement of the second connection interface.

In one or more embodiments, the system further comprises a first slit mechanism on the first vessel configured to allow external displacement of the first connection interface and configured to provide external displacement of the second connection interface. In one or more embodiments, the first slit mechanism is an aperture. In one or more embodiments, the first slit mechanism is a sealed aperture. In one or more embodiments, the seal of the sealed aperture is a frangible seal. In one ore more embodiments, the frangible seal is opened via a force of a user's hand.

In one or more embodiments, the system comprises a seal or a cover disposed between a vessel and a connection interface. In one or more embodiments, the seal or cover is frangible. In one or more embodiments, the seal or cover is pierceable.

In one or more embodiments, the system comprises a first seal or a first cover disposed between a first vessel and a first connection interface and a second seal or a second cover disposed between a second vessel and a second connection interface. In one or more embodiments, the first seal or first cover and a second seal or a second cover is frangible. In one or more embodiments, the first seal or first cover and a second seal or a second cover is frangible.

In one or more embodiments, the system further comprises a first hinge mechanism on a first vessel configured to provide external displacement of a first connection interface and comprising a second hinge mechanism on a second vessel configured to provide external displacement of a second connection interface.

In one or more embodiments, the system further comprises a piercing member on the first and/or second vessel configured to pierce a sealed opening of the first and/or second vessel, thereby allowing fluid to pass between the vessels. In one or more embodiments, the piercing member is a needle.

In one or more embodiments, the first and second connection interfaces are externally displaced via a sliding or a pulling motion.

In one or more embodiments, when the first connection interface and the second connection interface are displaced, the connection between the first vessel and the second vessel is decontaminated. In one or more embodiments, when the first and/or second connection interface slides off the first and/or second rail mechanism, the hermetic connection between the first vessel and the second vessel is maintained. In one or more embodiments, when the first and second connection interface slides off the first rail mechanism, the hermetic connection between the first vessel and the second vessel is maintained.

In one or more embodiments, the first connection interface and the second connection interface externally displace from the engagement between the first vessel and the second vessel through a slit or an aperture sealed by the force of the engagement between the first vessel and the second vessel.

In one or more embodiments, the first connection interface and the second connection interface externally displace from the engagement between the first vessel and the second vessel at or about the same time.

In one or more embodiments, the first vessel and/or the second vessel are selected from a vial, a bag, a bottle, a syringe, an infusion line, a connector, a filter, a manifold, a container port, a bag port, a bottle port, a vial port, and combinations thereof. In one or more embodiments, the interior of the container port may have a pressure less than the pressure of ambient air.

In one or more embodiments, circumferential ends of at least one of or both of the first and second connection interface ends are concealed within the hermetically sealed connection between the first vessel and the second vessel.

In one or more embodiments, the system further comprises a tab on at least one of the first connection interface and the second connection interface, the tab configured to allow for external displacement of the first connection interface and the second connection interface with respect to the first vessel and the second vessel. In one or more embodiments, the tab is configured to be pulled axially by a user, the axial pulling of the tab configured to displace the first connection interface and the second connection interface from the first vessel and the second vessel, while maintaining a hermetic seal between the first vessel and the second vessel.

In one or more embodiments, the first vessel and the second vessel are in fluid communication with one another after external displacement of the first connection interface and the second connection interface from the first vessel and the second vessel.

In one or more embodiments, the first connection interface is coupled to the first vessel and wherein the second connection interface is coupled to the second vessel.

In one or more embodiments, the first connection interface has edges configured to isolate an entire surface of the first connection interface that is exposed to contaminants from contacting an internal compartment of the first vessel and/or said second vessel when the first connection interface is engaged with the second connection interface.

In one or more embodiments, the first connection interface has edges configured to isolate a portion of the surface of the first connection interface that is exposed to contaminants from contacting an internal compartment of the first vessel and/or the second vessel when the first connection interface is engaged with the second connection interface.

In one or more embodiments, the edges are circumferential edges. In one or more embodiments, the circumferential edges define the outer portion of the first connection interface and the second connection interface. In one or more embodiments, the edges are further configured so the sides of the edges are not exposed to contaminants. In one or more embodiments, the sides of the edges that are exposed to an internal compartment of the first vessel and/or the second vessel are not exposed to contaminants.

In one or more embodiments, the outer portion of the edges of the first connection interface are not exposed to contaminants. In one or more embodiments, the edges of the first connection interface are configured to fully entrap the contaminants between the first and second connection interfaces upon engagement of the first and second connection interfaces. In one or more embodiments, the edges of the first connection interface are flat.

In one or more embodiments, the edges of the first connection interface are configured to snuggly fit the edges of the second connection interface. In one or more embodiments, the edge is an exterior portion of the connection interface that comes in contact with an interior compartment of the decontamination device and/or vessel.

In a further aspect, the present invention provides a method of decontaminating a connection between two vessels, the method comprising the steps of: providing a first connection interface attached to a first vessel; providing a second connection interface attached to a second vessel, engaging the first vessel to the second vessel; and externally displacing the first and second connection interfaces from the engagement between the first vessel and the second vessel, while a hermetically sealed connection between the first and second vessels is maintained.

In one or more embodiments, the step of externally displacing the first connection interface and the second connection interface occurs via a sliding motion.

In one or more embodiments, the step of externally displacing the first connection interface and the second connection interface occurs via sliding the first connection interface and the second connection interface axially with respect to the first vessel and the second vessel, thereby removing contaminants located between the first connection interface and second connection interface.

In one or more embodiments, an airtight seal is formed between the first connection interface and the second connection interface prior to the external displacement of the first connection interface and the second connection interface.

In one or more embodiments, the displacement of the first connection interface and the second connection interface is via a rail mechanism, such that the airtight sealed compartment between the first connection interface and the second connection interface slides off the rail.

In one or more embodiments, when the airtight seal between the first connection interface and the second connection interface is displaced, the connection between the first vessel and the second vessel is decontaminated.

In one or more embodiments, the airtight seal between the first connection interface and the second connection interfaces slides off the rail while maintaining the hermetically sealed connection between the first and second vessels.

In one or more embodiments, the step of externally displacing the first connection interface and the second connection interface occurs via peeling.

In one or more embodiments, the step of externally displacing the first connection interface and the second connection interface occurs at about the same time.

In one or more embodiments, circumferential ends of at least one of or both of the first and second connection interfaces ends are concealed within the airtight seal, allowing for sterile external displacement.

In one or more embodiments, the method further comprises connecting the first connection interface to the second connection interface via an engagement mechanism.

In one or more embodiments, connecting of the first connection interface to the second connection interface occurs prior to externally displacing the first and second connection interfaces from the engagement between the first vessel and the second vessel.

In one or more embodiments, the connecting of the first connection interface to the second connection interface is selected from a twisting of a thread onto a complementary thread, attaching ratchet teeth to a complementary retention member, and adhering a first connection interface to the second connection interface via an adhesive.

In one or more embodiments, the sealed aperture is a valve. In one or more embodiments, the valve is a one-way valve.

In a further aspect, the present invention provides a container housing a substance intended for intracorporal administration to a patient, the container comprising: a rail mechanism, and an externally displaceable connection interface, wherein the rail mechanism is configured to provide for the external displacement of the connection interface.

In one or more embodiments, the container further comprises an engagement mechanism. In one or more embodiments, the engagement mechanism is selected from a group consisting of a thread, a ratchet teeth mechanism, a snap-on mechanism and combinations thereof. In one or more embodiments, the connection interface is disposed on the rail mechanism. In one or more embodiments, the connection interface is disposed between rails of the rail mechanism. In one or more embodiments, the connection interface is attached to the rail mechanism via a frangible seal, wherein the frangible seal is configured to break/rupture by a force of a user's hand upon displacement of the connection interface from the rail. In one or more embodiments, the connection interface is attached to the container via a frangible seal, wherein the frangible seal is configured to break/rupture by a force of the user's hand upon displacement of the connection interface from the rail. In one or more embodiments, the connection interface may displace externally between the teeth of a ratchet teeth engagement mechanism. In one or more embodiments, the connection interface may displace externally between the threads of a thread/luer engagement mechanism.

In one or more embodiments, the container has a flexible wall. In one or more embodiments, the container has a rigid wall.

In one or more embodiments, the container is a bag. In one or more embodiments, the container is a bottle. In one or more embodiments, the container is a vial. In one or more embodiments, the container is a syringe.

In one or more embodiments, the connection interface displaces between teeth of a ratchet teeth engagement mechanism. In one or more embodiments, the connection interface displaces between threads of a thread engagement mechanism. In one or more embodiments, the connection interface has an adhesive on a surface of the connection interface.

In one or more embodiments, the connection interface has at least one of a disinfecting agent and a sterilizing agent disposed on the surface of the connection interface.

In yet a further aspect, the present invention provides a decontamination connector for decontaminating a connection between vessels, comprising: a port; a connection interface configured to cover the port, wherein the connection interface is configured to displace externally from the port; an engagement mechanism, wherein the engagement mechanism is configured to provide coupling between the port and a vessel; and wherein following the coupling, the decontamination connector and the vessel form a hermetically coupled connection, while the connection interface is externally displaced from the port while maintaining the hermetically coupled connection between the decontamination connector and the vessel.

In one or more embodiments, the connection interface is selected from the group consisting of a film, a plastic, a polymer, or a metal alloy.

In one or more embodiments, the connection interface is configured to seal the port of a first vessel.

In one or more embodiments, an engagement mechanism provides a secure engagement between the decontamination connector and the second vessel. In one or more embodiments, the engagement mechanism is selected from a thread, a luer, an adhesive, and a ratchet teeth mechanism. In one or more embodiments, the engagement mechanism is configured to form an airtight connection between the first vessel and the second vessel.

In one or more embodiments, the connection forms a sealed aperture. In one or more embodiments, the sealed aperture provides for the displacement of the connection interface through the sealed aperture while maintaining the hermetically coupled connection between the decontamination connector and the vessel.

In one or more embodiments, the connector further comprises an engagement mechanism configured to provide an engagement between the connection interface and a second connection interface on a second vessel.

In one or more embodiments, the engagement mechanism is selected from a thread, a luer, an adhesive, and a ratchet teeth mechanism. In one or more embodiments, the engagement mechanism includes a mechanical object that connects the first connection interface and a second connection interface on a second vessel providing for a simultaneous external displacement of the first and second connection interfaces. In one or more embodiments, the mechanical object is a protrusion, a dent or a hook.

In one or more embodiments, the connector further comprises a rail mechanism configured to provide for the external displacement of the connection interface. In one or more embodiments, the rail mechanism is configured to provide for the external displacement of the connection interface via a sliding motion.

In one or more embodiments, the connection interface displaces externally from the port via a hinge mechanism. In one or more embodiments, the connection interface displaces externally from the port via peeling.

In one or more embodiments, when the connection interface externally displaces from the port, the hermetic connection between the decontamination connector and the vessel is maintained.

In one or more embodiments, the decontamination connector is attached to or integrally attached to a vessel, to a container or to an administration device. In one or more embodiments, the container is selected from a vial, a bag, and a bottle. In one or more embodiments, the vessel is selected from a syringe, an infusion line, a connector, a filter, and a manifold.

In one or more embodiments, the port is selected from the group consisting of a container port, a syringe port, an infusion line port, a connector port, a manifold port, and a vessel port.

In one or more embodiments, the circumferential end of the connection interface end is concealed within the hermetically coupled connection between the decontamination connector and the vessel.

In one or more embodiments, the decontamination connector further comprises a tab, the tab configured to allow for the external displacement of the connection interface with respect to the port of the decontamination connector.

In one or more embodiments, the tab is configured to be pulled axially by a user, the axial pulling of the tab configured to displace the connection interface from the port of the decontamination connector while maintaining a hermetic seal between the decontamination connector and the vessel.

In one or more embodiments, the port is in fluid communication with the vessel after external displacement of the connection interface from the port.

In one or more embodiments, the connection interface is coupled to the port of the decontamination connector.

In one or more embodiments, the decontamination connector further comprises a piercing member configured to pierce a seal or cover of a vessel or a container.

In one or more embodiments, the decontamination connector further comprises a valve. In one or more embodiments, the valve is a one-way valve.

In yet a further aspect, the present invention provides a decontamination connector for the decontamination of a connection between vessels, comprising: a port; a connection interface configured to cover the port, wherein the connection interface is configured to displace externally from the port; a rail mechanism configured to provide for the external displacement of the connection interface; and an engagement mechanism, wherein the engagement mechanism is configured to provide coupling between the port and a vessel, wherein following the coupling, the decontamination connector and the vessel form a hermetically coupled connection, while the connection interface is externally displaced from the port via the rail mechanism while maintaining the hermetically coupled connection between the decontamination connector and the vessel.

In yet a further aspect, the present invention provides a decontamination connector for the decontamination of a connection between vessels, comprising: a port; a connection interface configured to cover the port, wherein the connection interface is configured to displace externally from the port; a sealed aperture configured to provide for the external displacement of the connection interface; and an engagement mechanism, wherein the engagement mechanism is configured to provide coupling between the port and a vessel, wherein following coupling, the decontamination connector and the vessel form a hermetically coupled connection, while the connection interface is externally displaced from the port through the sealed aperture while the hermetically coupled connection between the decontamination connector and the vessel is maintained.

In one or more embodiments, the sealed aperture reseals at about the time of external displacement of at least one of the connection interfaces of the decontamination connector and the vessel. In one or more embodiments, the sealed aperture reseals prior to detachment of at least one of the connection interfaces of the decontamination connector and the vessel from the decontamination connector and the vessel.

In yet a further aspect, the present invention provides a method of decontaminating a connection between two vessels, the method comprising the steps of: providing a first connection interface attached to a first vessel; providing a second vessel; engaging the first vessel to the second vessel; and externally displacing the first connection interface from the engagement between the first vessel and the second vessel, while a hermetically sealed connection between the first and second vessel is maintained.

In one or more embodiments, the step of externally displacing the connection interface occurs via a sliding motion. In one or more embodiments, the step of externally displacing the connection interface occurs via sliding axially with respect to the first vessel and the second vessel, thereby removing contaminants located on the first connection interface.

In one or more embodiments, displacement of the connection interface is via a rail mechanism. In one or more embodiments, when the connection interface is displaced, the connection between the first vessel and the second vessel is decontaminated. In one or more embodiments, the connection interface slides on the rail while maintaining the hermetically sealed connection between the first and second vessels.

In one or more embodiments, the step of externally displacing the connection interface occurs via peeling.

In one or more embodiments, the method further comprises connecting the first vessel to the second vessel via an engagement mechanism. In one or more embodiments, connecting of the first vessel to the second vessel occurs prior to externally displacing the connection interface from the engagement between the first vessel and the second vessel.

In one or more embodiments, connecting of the first vessel to the second vessel is selected from a twisting of a thread onto a complementary thread, attaching ratchet teeth to a complementary retention member, and adhering the first vessel to the second vessel via an adhesive.

In yet a further aspect, the present invention provides a vessel with a rail mechanism.

In one or more embodiments, the vessel is a container. In one or more embodiments, the vessel has a flexible wall. In one or more embodiments, the vessel has a rigid wall. In one or more embodiments, the vessel is a bag or a bottle. In one or more embodiments, the vessel is a vial. In one or more embodiments, the vessel is a syringe. In one or more embodiments, the rail mechanism is attached to the vessel. In one or more embodiments, the rail mechanism is integrally attached to the vessel. In one or more embodiments, the rail mechanism forms a unitary structure with the vessel.

In yet a further aspect, the present invention provides a vessel having a rail mechanism and a vessel surface disposed on the rail mechanism. In one or more embodiments, the vessel surface is disposed between the rails of the rail mechanism. In one or more embodiments, the rail mechanism is configured to provide for the sliding of the vessel surface along the rail mechanism.

Unless otherwise defined, all technical or/and scientific terms used herein have the same meaning as commonly understood by one of ordinary skill in the art to which the invention pertains. Although methods and materials similar or equivalent to those described herein can be used in the practice or testing of embodiments of the invention, exemplary methods or/and materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1D are schematic illustrations presenting an exemplary decontamination system which comprise a first connection interface and a second connection interface configured to engage with each other and to allow a hermetic and decontaminated communication between a first vessel and a second vessel, according to some embodiments of the invention.

FIG. 2 is a magnified cut view of the first connection interface and second connection interface shown in FIGS. 1A-1D, the connection interfaces configured to seal an opening of a first vessel and a second vessel, respectively, according to some embodiments of the invention.

FIGS. 3A-3C are schematic back cut views of a first connection interface and a second connection interface, each of the connection interfaces configured to seal an opening within a corresponding vessel, according to some embodiments of the invention.

FIGS. 4A-4D are schematic top cut views of a vessel having an opening for allowing fluid flow therethrough and an aperture to accommodate a connection interface, wherein prior to externally displacing from the vessel, the connection interface is configured to seal the opening and aperture (FIG. 4A), wherein when the connection interface externally displaces from the opening and aperture (FIGS. 4B-4C) and following the external displacement (FIG. 4D), the opening allows the fluid to flow therethrough and the aperture reseals allowing a hermetic engagement between vessels, according to some embodiments of the invention.

FIGS. 5A-5E are schematic side cut views of a first connection interface and a second connection interface attached to medical vessels via ports, the connection interfaces configured to engage with each other and externally displace from the vessel via a rail mechanism, according to some embodiments of the invention.

FIGS. 6A-6E are schematic illustrations presenting a further exemplary decontamination system with an engagement mechanism configured to provide a secure engagement between two vessels, according to some embodiments of the invention.

FIGS. 7A-7D are schematic illustrations presenting a further exemplary decontamination system with an engagement mechanism configured to provide a secure engagement between connection interfaces, according to some embodiments of the invention.

FIGS. 8A-8B are schematic back cut views showing a first connection interface and a second connection interface, wherein circumferential ends of at least one of the connection interfaces are concealed within the airtight seal formed following engagement of the connection interfaces, according to some embodiments of the invention.

FIGS. 9A-9B are schematic back cut views of a first connection interface and a second connection interface attached to a first vessel and a second vessel, respectively, the connection interfaces may externally displace from the first and second vessels, via a rail mechanism, according to some embodiments of the invention.

FIG. 10A-10B are schematic back cut views of a first and a second connection interfaces attached to a first and a second vessel, respectively, the connection interfaces are different in their exterior surface area and are configured to externally displace from the first vessel and the second vessel, via a rail mechanism, according to some embodiments of the invention.

FIGS. 11A-11E are schematic illustrations presenting connection interfaces configured to be coupled to and allow an engagement between various types of medical vessels and devices, according to some embodiments of the invention.

FIG. 12 is a schematic illustration presenting a first vessel attached to a plurality of connection interfaces wherein each connection interface is configured to engage with a complementary connection interface present on a medical vessel or device, according to some embodiments, of the invention.

FIGS. 13A-13C are schematic illustrations presenting a further exemplary decontamination connector which includes a connection interface configured to cover a port, the connection interface is further configured to displace externally from the port, according to some embodiments of the invention.

FIGS. 14A-14D are schematic illustrations presenting a decontamination connector which allows sterile engagement of vessels, according to some embodiments of the invention.

FIGS. 15A-15C are schematic back view illustrations presenting a decontamination connector which allows sterile engagement of vessels, according to some embodiments of the invention.

FIGS. 16A-16C are schematic illustrations presenting a first and a second medical vessel attached to connection interfaces, wherein a decontamination connector mediates the engagement and fluid passageway between the vessels, according to some embodiments of the invention.

FIG. 17 is a schematic illustration presenting a system wherein various types of medical vessels or devices may be attached to connection interfaces, and wherein a decontamination connector mediates the engagement and fluid passageway between the vessels, according to some embodiments of the invention.

FIGS. 18A-18D are schematic illustrations presenting yet another exemplary decontamination system which comprises a first connection interface and a second connection interface configured to engage with each other and to allow a hermetic and decontaminated communication between a first vessel and a second vessel, wherein the connection interfaces externally displace by a hinge mechanism, according to some embodiments of the invention.

FIGS. 19A-19C are schematic top cut views of a vessel's port having a connection interface which can externally displace by a hinge mechanism, according to some embodiments of the invention.

FIGS. 20A-20E are schematic side cut views presenting yet another exemplary decontamination system which comprises a first connection interface and a second connection interface and a piercing member and a valve disposed within at least the port of first medical vessel, according to some embodiments of the invention.

FIG. 21 is a schematic illustration presenting yet another exemplary decontamination system which comprises a first connection interface and a second connection interface and a piercing member disposed within at least the port of first medical vessel, the piercing member is actuated by a twist motion, according to some embodiments of the invention.

FIG. 22 is a schematic illustration presenting yet another exemplary decontamination system which comprises a first and a second connection interfaces and a piercing member disposed within at least the port of first medical vessel, the piercing member is actuated by a downward pushing motion, according to some embodiments of the invention.

FIGS. 23A-23D are schematic side cut views presenting yet another exemplary decontamination system which comprises a first connection interface and a second connection interface which are in a folded position and peeled off while externally displacing from the openings of a first vessel and a second vessel, according to some embodiments of the invention.

FIGS. 24A-24B are schematic illustrations presenting yet another exemplary decontamination system comprising a first connection interface and a second connection interface configured to engage with each other and to allow a hermetic and decontaminated communication between vessels, wherein a click sound is heard at about the time of engagement of the vessels, according to some embodiments of the invention.

FIGS. 25A-25B are schematic illustrations presenting yet another exemplary decontamination system comprising a first connection interface and a second configured to engage with each other and to allow a hermetic and decontaminated communication between vessels, wherein a click sound is heard at about the time of external displacement of the vessels, according to some embodiments of the invention.

It should be appreciated that for simplicity and clarity of illustration, elements shown in the figures have not necessarily been drawn to scale. For example, the dimensions of some of the elements are exaggerated relative to each other for clarity. Further, where considered appropriate, reference numerals have been repeated among the figures to indicate corresponding elements.

### DETAILED DESCRIPTION OF THE INVENTION

It is understood that the invention is not limited to the particular methodology, devices, items or products etc., described herein, as these may vary as the skilled artisan will recognize. It is also to be understood that the terminology used herein is used for the purpose of describing particular embodiments only and is not intended to limit the scope of the invention. The following exemplary embodiments may be described in the context of exemplary bedding articles for ease of description and understanding. However, the invention is not limited to the specifically described products and methods and may be adapted to various applications without departing from the overall scope of the invention. All ranges disclosed herein include the endpoints. The use of the term “or” shall be construed to mean “and/or” unless the specific context indicates otherwise.

The present invention relates to devices, methods and systems allowing an engagement of medical containers or devices in a sterile manner. In one embodiment, the present invention provides connection interfaces, that may be coupled to, or integrally formed with, vessels or containers, such as vials, syringes, etc. The invention provides a solution to an unmet and long felt need in the medical setting and allows connecting in a sterile manner, two or more vessels. The herein disclosed devices and systems are user friendly, cost effective and abolish the need for complicated and expensive known methods for transferring medical substances. In an embodiment of the invention, the herein disclosed devices and systems are disposable. In an embodiment of the invention, the herein disclosed devices and systems are non-disposable. In an embodiment of the invention, the herein disclosed vessels and devices are intended for one-time use. In an embodiment of the invention, the herein disclosed vessels and devices are intended to be reused.

In an aspect of the invention, the devices and systems include a first connection interface attached to a first vessel and a second connection interface attached to a second vessel. The first and second connection interfaces are configured to allow an engagement therebetween the connection interfaces. When the connection interfaces engage each other, ambient air particles present at the vicinity thereof are entrapped within/between the engagement formed by the connection interfaces. The engagement between the connection interfaces further establishes an airtight engagement of the vessels. The connection interfaces are further configured to externally displace from the vessels, thereby allowing fluid flow between the vessels and contaminants-free connection of the vessels.

Accordingly, the herein disclosed invention allows transferring medical substances in a contaminants-free, or in a substantially contaminants-free manner. In one or more embodiments, the herein disclosed invention allows connecting vessels in a contaminants-free, or in a substantially contaminants-free manner.

In one or more embodiments, the herein disclosed invention provides a fluidic passageway or communication between medical containers in a contaminants-free, or in a substantially contaminants-free manner.

In one or more embodiments, the herein disclosed invention affords to isolate and/or entrap ambient air particles present between two, or more, vessels. In one or more embodiments, the herein disclosed invention, allows to substantially decrease the chances to introduce a contamination within a medical substance, when preparing or administering medical substances.

As used herein, the term “substances” refers to various types of materials that should be kept sterile. The substances may be liquid, solid, semi-solid, or gas. In one or more embodiments, the substances are “medical substances”. As used herein the term “medical substances” refers to and encompasses any of the various medical drugs, fluids, nutritional products, solid powders, suspensions, liquids solutions and the like.

As used herein, the term “contaminant-free” is interchangeable with the term “sterile”, “disinfected”, and “decontaminated”. The term refers to substances that are free or substantially free of ambient air particles and/or pathogens, including but not limited to dirt, bacteria, viruses, funguses, spores, and/or pyrogens. Typically, when less or no air is introduced within medical substances, the chances of contamination by pathogens, such as, bacteria, viruses, funguses, spores, pyrogens or the alike is completely abolished or significantly reduced.

As used herein, “substantially contaminants-free” means significantly less ambient air present when transferring medical substances with the herein disclosed vessels and systems, as compared to comparable conditions for transferring medical substances without the herein disclosed vessels and systems.

As used herein, the term “ambient air particles” is interchangeable with the term “environmental air particles” and refers to air particles present in a non-filtered or filtered environment. For example, air can be purified by filters, such as a High Efficiency Particulate Air (HEPA) filter.

As used herein, the term “connection interface” encompasses any surface, layer plane or the alike that can be attached to a vessel. The term may encompass a structure that can be coupled to a vessel and that can engage with a complementary connection interface. In an embodiment of the invention, the connection interface may seal or cover an aperture of a vessel. In an embodiment of the invention, the connection interface may seal or cover an opening for fluid passageway of a vessel. In an embodiment of the invention, the connection interface may be a surface of a container. In an embodiment of the invention, the connection interface may be a surface of a device. In an embodiment of the invention, the connection interface may be a surface of a vessel. In an embodiment of the invention, the connection interface abuts a container. In an embodiment of the invention, the connection interface is surface mounted to a container. In an embodiment of the invention, the connection interface is surface mounted to a wall of the container. In an embodiment of the invention the connection interface is flush mounted to a container.

In an embodiment of the invention, the connection interface abuts a device. In an embodiment of the invention, the connection interface is surface mounted to a device. In an embodiment of the invention, the connection interface is surface mounted to a wall of a device. In an embodiment of the invention the connection interface is flush mounted to a device.

In an embodiment of the invention, the connection interface abuts a vessel. In an embodiment of the invention, the connection interface is surface mounted to a vessel. In an embodiment of the invention, the connection interface is surface mounted to a wall of a vessel. In an embodiment of the invention the connection interface is flush mounted to a vessel. In an embodiment of the invention, the connection interface is flush mounted to a wall of a vessel. In one or more embodiments, a plurality of connection interfaces abuts a vessel and/or device. In an embodiment of the invention, a plurality of connection interfaces is surface mounted to a vessel. In an embodiment of the invention, a plurality of connection interfaces is surface mounted to a at least one wall of a vessel. In an embodiment of the invention, a plurality of connection interfaces is surface mounted to a vessel. In an embodiment of the invention, a plurality of connection interfaces is flush mounted to a vessel. In an embodiment of the invention, a plurality of connection interface is flush mounted to at least one wall of a vessel. In one or more embodiments, a plurality of connection interfaces is attached to a vessel, a container, and/or a device.

In an embodiment of the invention, the present invention discloses a first connection interface and a second connection interface. The first connection interface and the second connection interface may be similar or may be different in size and/or shape. In one or more embodiments, the exterior surface area facing ambient air, of the connection interfaces may be equal. In one or more embodiments, the exterior surface area facing ambient air, of the connection interfaces may be non-equal. In one or more embodiments, the difference in surface area between the exterior surface area of a plurality of connection interfaces is less than 0.3 inches squared. For example, less than 0.2 inches squared, or less than 0.1 inches squared.

In an embodiment of the invention, the first connection interface and the second connection interface when engaged with each other entrap ambient air particles therebetween. In an embodiment of the invention, the first connection interface and the second connection interface when engaged with each other externally displace from a vessel(s), and/or from the connection interface(s).

In one or more embodiments, the connection interface has a thin film layer. In an exemplary embodiment, the connection interface may present a thickness of about 1 mm or below. Alternatively, the connection interface may present a thickness of above 1 mm. The connection interface may be manufactured from various materials, including, an elastic polymer. The connection interface may be made from a plastic material. The connection interface may be a rubber or a polymer. The connection interface may be formed of a metal material (e.g., a metal alloy). In yet a further exemplary embodiment, the connection interface may preset the form of a thin film made of an aluminum foil, cling wrap, or the alike. The metal alloy may include, for example, aluminum. In yet a further exemplary embodiment, the connection interface may preset the form of a thin film made of an aluminum foil, or the alike. In one or more embodiments, the connection interface is attached to, or is integrally formed with a vessel, or port thereof. In one or more embodiments, the connection interface is peelable from the vessels. In one or more embodiments, the peelable connection interface is attached to a vessel via an adhesive. In one or more embodiments, the peelable connection interface may be detached from a vessel surface by the force of a user's hand. In one or more embodiments, the connection interface is movable or slidable along a rail located along a length of the aperture of a vessel. In one or more embodiments, the first and second connection interfaces may be attached to or engage each other via various mechanisms. For example, the connection interfaces may be engaged via an engagement mechanism, such as a thread, a luer, an adhesive, a mechanical object, a dent and a ratchet teeth mechanism. In one or more embodiments, the connection interface may be covered by a cover (not shown). In one or more embodiments, the cover may be a peelable cover. In one or more embodiments, a sterilizing or disinfecting substance may be located between the connection interface and the cover. In one or more embodiments, the sterilizing or disinfecting substance may be an alcohol. In one or more embodiments, the alcohol may be 70% isopropyl alcohol.

The connection interfaces may be coupled within any portion of the vessel. For example, the connection interfaces may be connected onto the vessels' body or aperture, the vessels' wall or port thereof.

In one or more embodiments, the first connection interface and the second connection interface are configured to prevent contaminants from entering the fluid passageway between a first vessel and a second vessel. In one or more embodiments, the first connection interface and the second connection interface are configured to prevent contaminants from entering the interior of the vessel's body. In one or more embodiments, the first connection interface and the second connection interface are configured to prevent contaminants from entering a fluid housed inside the vessel's body.

In one or more embodiments, a first connection interface and a second connection interface are configured to prevent contaminants from contacting an internal compartment of the first vessel and/or the second vessel.

As used herein the term “external displacement” refers to a displacement (i.e., dislocation) of the herein disclosed first and/or second, and/or any additional connection interfaces. In an embodiment of the invention, the displacement is external, namely, outside the vessels being connected by the herein disclosed system. In an embodiment of the invention, the displacement is external to the fluid communication established following engagement of the vessels being connected by the herein disclosed system, methods and devices. In an embodiment of the invention, the displacement maintains a hermetic seal of the connection interfaces and/or the vessels. The displacement may occur via a sliding motion, or by a pulling out motion, or by peeling the connection interfaces. In an embodiment of the invention, the displacement occurs for both the first connection interface and the second connection interface. The external displacement may optionally occur simultaneously for both connection interfaces or may occur consecutively. In an embodiment of the invention, the external displacement established a fluid passageway between two or more vessels. In one or more embodiments, the connection interface is configured to hermetically seal an aperture present in a vessel. In one or more embodiments, at about the time of external displacement of the connection interfaces, the aperture through which the connection interfaces pass through reseals, allowing a hermetic airtight connection between two or more vessels. In one or more embodiments, after external displacment of the connection interfaces, the aperture through which the connection interfaces pass through reseals, allowing a hermetic airtight connection between two or more vessels. In one or more embodiments, the aperture through which the connection interfaces pass through reseals prior to detachment of the connection interfaces from the vessels.

As used herein the term “vessel” refers to any device utilized for containing, housing or transferring substances as herein disclosed. In one or more embodiments, the vessel may be used for containing medical substances. In an embodiment of the invention, the vessel is a medical vessel. In an embodiment of the invention, the vessel is a medical device. In an embodiment of the invention, the vessels are used for, and adapted to allow connection to another vessel(s). In an embodiment of the invention, the vessel may be a medical container utilized for accommodating medical substances. Various types of medical containers are contemplated. The medical container may be selected, without limitation, from a vial, a bag, a chamber, a housing, a bottle, and the alike. In an embodiment of the invention, the term vessel further encompasses elements that can be used to connect between vessels. In accordance with this embodiment, the vessel may be selected, without limitation, from a connector, a port, a syringe, an infusion line, a tubing, a syringe, a filter, a spike, a port and a manifold. In an embodiment of the invention, one or more connection interfaces (for example, two or more, three or more, etc.) may be coupled to a first vessel and each of those connection interfaces may be coupled and engage with a one or more connection interfaces present on a one or more vessels. In one or more embodiments, the vessel may be a container, a bag, a bottle, a connector, an infusion line or a syringe having a plurality of externally displaceable connection interfaces. In one or more embodiments, the vessels to be engaged may have similar surface area or similar contact surface area (i.e., surface onto which the connection interface is coupled to). For example, a first vessel and a second vessel may have similar surface area or similar contact surface area. In one or more embodiments, the vessels to be engaged may have different surface area or different contact surface area. For example, a first connection interface may have a greater surface area or contact surface area than a second vessel. As used herein the term “fluid communication” refers to two vessels in which substances may pass therethrough either directly or indirectly. The fluid communication may occur via a fluid passageway that allows for the flow/transfer of a medical substance.

The herein systems and connectors may include one or more valves that restrict fluid flow or passageway. For example, one or more valves may be located at the opening of one or more vessels. In a further exemplary embodiment one or more valves may be disposed at the aperture(s) of the vessels or at a location within a port or vessel body of the vessels. The valves are generally provided to seal and prevent fluid flow prior to any engagement of vessels and/or external displacement of connection interfaces. In an exemplary embodiment, when valves are provided to seal an aperture that provides for the external displacement of connection interfaces, the valves may establish resealing of the apertures at about the time or following the external displacement of the connection interfaces, preventing any leakage of fluids from the engagement between the vessels. In certain embodiments, the resealing occurs at about the time or prior to the external displacement. In certain embodiments, the valves that provide for the passage and external displacement of the connection interfaces may be made of a rubber material, an elastomeric material, a plastic material, and combinations thereof. In an embodiment of the invention, the valve or aperture providing for the passage and external displacement of at least one or two connection interfaces maintains a hermetic seal between at least two vessels or at least two devices by a force of the engagement between at least two vessels or devices. In some embodiments the force of engagement that maintains the hermetic seal between two vessels or devices during the external displacement of the connection interfaces is provided by an engagement mechanism selected from a thread, a luer, a ratchet teeth mechanism, a snap-on mechanism, and combinations thereof. In certain embodiments, the aperture, sealed aperture, or valve providing for the passage and external displacement of at least one or at least two connection interfaces reseals prior to the connection interfaces fully displacing from the first and/or second vessel or device. In some embodiments, the displacement of the connection interfaces occurs in the space between two ratchet teeth. In certain embodiments, the displacement of the connection interfaces occurs between threads of a thread/luer engagement mechanism. In certain embodiments, the valves may prevent backflow of fluids. In some embodiments, the valves may be one-way valves.

In an embodiment of the invention, the herein disclosed devices, systems and methods allow fluid communication in contaminant-free, or substantially contaminant-free manner. For example, between three or more, four or more, five or more or six or more vessels. In an embodiment of the invention, the herein disclosed devices, systems and methods allow fluid communication between three, four, five, six, or seven vessels. For example, the devices, systems, and methods of the invention allow fluid communication between two containers, between two bags, between a syringe and a bag, between a connector and a bag, between a vial and a bag, between a vial and a bottle, between a syringe and a vial or between a syringe and a connector.

A further purpose of this invention is to provide a connection interface that reduces the presence of non-purified air and/or air particles. This invention focuses on connection interfaces that are substantially reduced of contaminants and ambient air particles and/or entirely contaminant free and entirely free of ambient air particles.

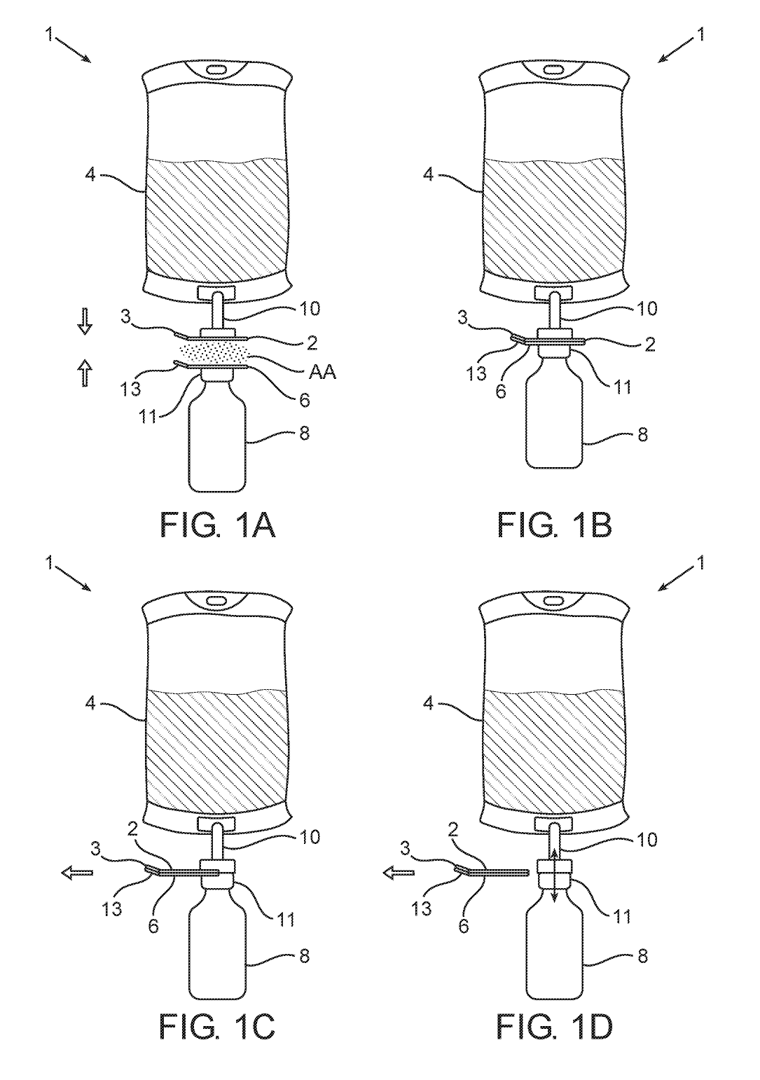

Referring now to the drawings, FIGS. 1A-1D illustrate an exemplary decontamination system 1 for the engagement of one or more vessels. Decontamination system 1 includes a first connection interface 2 attached to a first vessel 4. Decontamination system 1 further includes a second connection interface 6 attached to a second vessel 8. First connection interface 2 may be provided when coupled to first vessel 4, and second connection interface 6 may be provided when coupled to second vessel 8. Optionally, the connection interface 2 may be integrally manufactured with vessel 4 and/or connection interface 6 may be integrally manufactured with vessel 8. Further optionally, the connection interfaces may be manufactured or assembled separately from the vessels and coupled thereto at a later stage in a sterile manner. First connection interface 2 and second connection interface 6 may be similar or may be different in size and/or shape. First connection interface 2 and/or second connection interface 6 may be in the form of a film. The film may be manufactured from, for example, an elastic polymer (e.g., a rubber) or from a metal film (e.g., a metal alloy) or polymer and may be of a thickness of less than about 5 millimeters. As presented in FIGS. 1A-1D, first vessel 4 is a bag, such as, an infusion bag, and second vessel 8 is a bottle. It is to be noted that other forms of vessels or containers are herein contemplated. For example, the vessel may be a medical container chosen from a vial, a bag, a bottle, and the alike. The vessel may also be chosen from a syringe, an infusion line, a connector, a filter, a manifold, a bag port, a bottle port, a vial port, or any of the alike. First vessel 4 includes port 10 which is attached to first connection interface 2. Vessel 8 also includes port 11 which is attached to second connection interface 6. In accordance with this embodiment, first connection interface 2 can be used to seal and/or cover port 10 of first vessel 4. Second connection interface 6 can be used to seal and/or cover port 11 of second vessel 8. Optionally, first connection interface 2 may be directly attached to first vessel 4 without a port (not shown) and/or second connection interface 6 may be directly attached to second vessel 8 without a port (not shown). In accordance with this embodiment, first connection interface 2 may abut, may be flush mounted or may be surface mounted to first vessel 4 and/or second connection interface 6 may abut, may be flush mounted or surface mounted to second vessel 8.

First connection interface 2 and second connection interface 6 are configured to allow for an engagement between first vessel 4 and second vessel 8.

FIG. 1A shows decontamination system 1 of the invention prior to engagement of the connection interfaces 2 and 6, wherein ambient air AA particles surround the connection interfaces 2 and 6. FIG. 1B shows decontamination system 1 of the invention following an engagement between first and second connection interfaces 2 and 6, respectively. An engagement between first connection interface 2 and second connection interface 6 involves an entrapment of ambient air AA particles present therebetween the connection interfaces 2 and 6. FIG. 1C shows decontamination system 1 during external displacement of first and second connection interfaces 2 and 6, respectively.

Decontamination system 1 may further comprise a tab 3 positioned on a longitudinal end of first connection interface 2, and a tab 13 positioned on a longitudinal end of second connection interface 6. Tabs 3 and 13 are configured to allow for an external displacement of first connection interface 2 and second connection interface 6 with respect to first vessel 4 and second vessel 8, respectively. The tabs 3 and 13 are configured to be pulled out axially by a user, the axial pulling of the tabs 3 and 13 is configured to allow for a displacement of first connection interface 2 and second connection interface 6 from the first vessel 4 and second vessel 8, respectively. FIG. 1D shows decontamination system 1 following external displacement of first and second connection interfaces 2 and 6, respectively. The herein disclosed decontamination system 1 establishes a hermetically sealed connection of the two vessels which is maintained during the external displacement of the connection interfaces and as long as the vessels remain in contact. In an embodiment of the invention, the engagement between the first and second vessel 4, and 8, respectively, results in an ambient air AA particles-free engagement between the vessels. Ambient air AA interfacing the first and second interfaces is entrapped by the engaged first connection interface 2 and the second connection interface 6. In an embodiment of the invention, the first and second vessels 4 and 8, respectively are in fluid communication with one another at about the time or after the external displacement of the first and second connection interfaces 2 and 6, respectively.

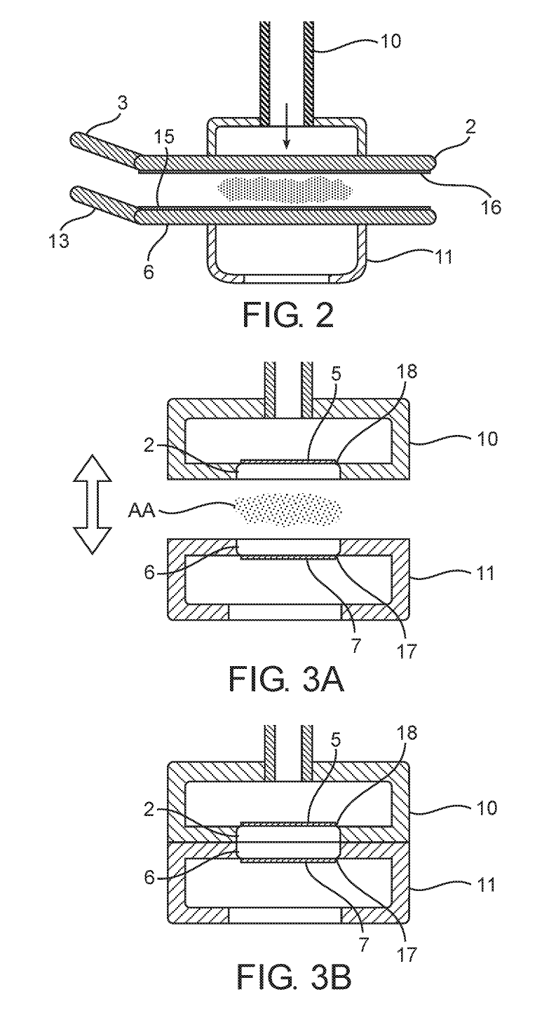

FIG. 2 is a magnified side cut view of the connection interfaces 2 and 6, and of ports 10 and 11 of vessels 4 and 8, respectively. The one or both connection interfaces 2 and 6 may include an engagement mechanism in the form of adhesive tapes 15 and 16, respectively. The adhesive tapes 15 and 16 may be a thin layer of an adhesive film/tape which may be covered, prior to use thereof, by a cover tape (not shown). The herein disclosed system may include, in an alternative embodiment, an adhesive tape, 16 or 15 which may be present merely on one of the interfaces 2 or 6, respectively. The adhesive film/tape, 15 or 16, and/or cover tape (not shown) may have a sterilizing or disinfecting substance on the film/tape or on the cover tape.

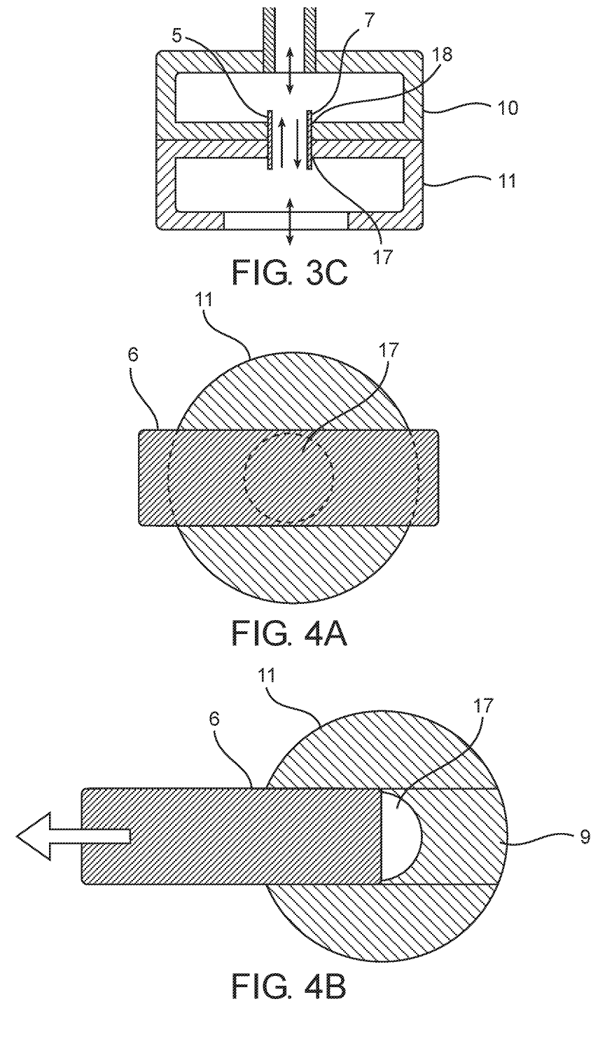

Reference is now made to FIGS. 3A-3C which show back cut views of the connection interfaces 2 and 6 attached to ports 10 and 11, respectively. Connection interfaces 2 and 6 are configured such that, while present, they seal openings 18 and 17 of vessels 4 and 8, respectively. Openings 18 and 17 may be covered by valve 5 and valve 7, respectively. Valves 5 and 7 are closed while the interfaces seal the openings 18 and 17 and are opened upon external displacement of the interfaces 2 and 6, respectively. FIG. 3A presents ports 10 and 11, wherein openings 18 and 17 are sealed by the connection interfaces 2 and 6, respectively. Following engagement of the interfaces (shown in FIG. 3B), the interfaces may be externally displaced. FIG. 3C shows the ports 10 and 11 following the external displacement. The ports 10 and 11 are securely engaged with each other and the openings 17 and 18 being uncovered by valves 5 and 7, respectively and remained as such, allowing fluid passageway and communication between the vessels 4 and 8.

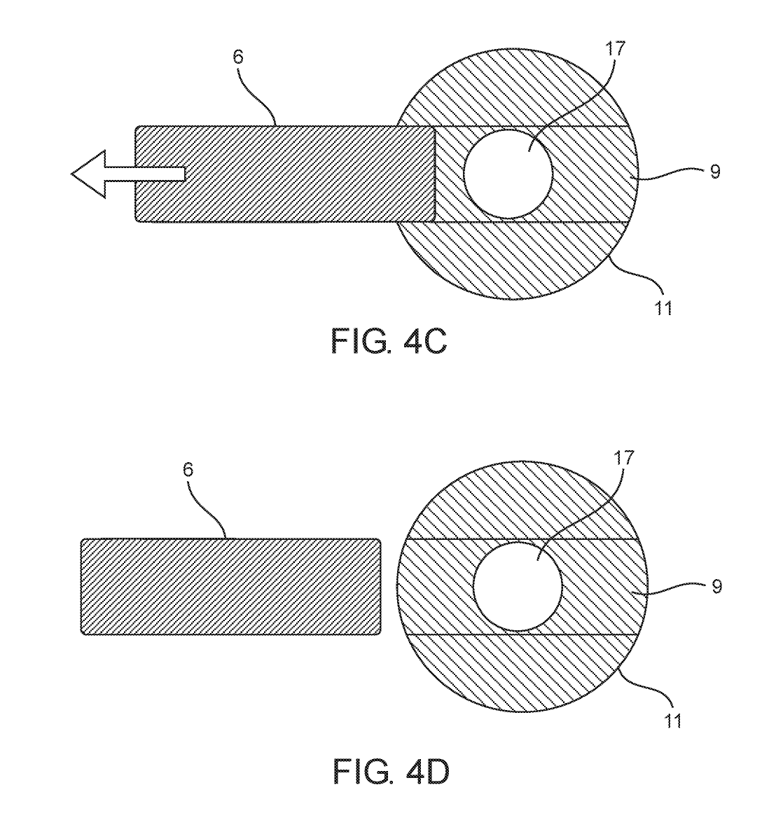

FIGS. 4A-4D show top views of connection interface 6 of system 1. The connection interface 6 presents a rectangular-like shape. FIG. 4A illustrates the connection interface 6 when positioned along a diameter of port 11 and sealing opening 17 and/or aperture 9. Aperture 9 is configured to allow the accommodation of connection interface 6 prior to the external displacement. When present, the connection interface 6 seals aperture 9. FIGS. 4B-4C depict the connection interface 6 during the external displacement. FIG. 4C depicts aperture 9 resealing at about the time of external displacement of connection interface 6. FIG. 4D depicts the connection interface following the external displacement, aperture 9 reseals and opening 17 is exposed enabling fluid flow between vessels 4 and 8. It is to be noted that aperture 9 may be made of a elastomeric, rubber or plastic material which may facilitate the resealing of aperture 9 at about the time of external displacement of connection interface 6. It is also to be noted that aperture 9 reseals at about the time of external displacement of connection interface 6 due to the force of engagement between vessels 4 and 8 or due to the force of engagement between ports 10 and 11.

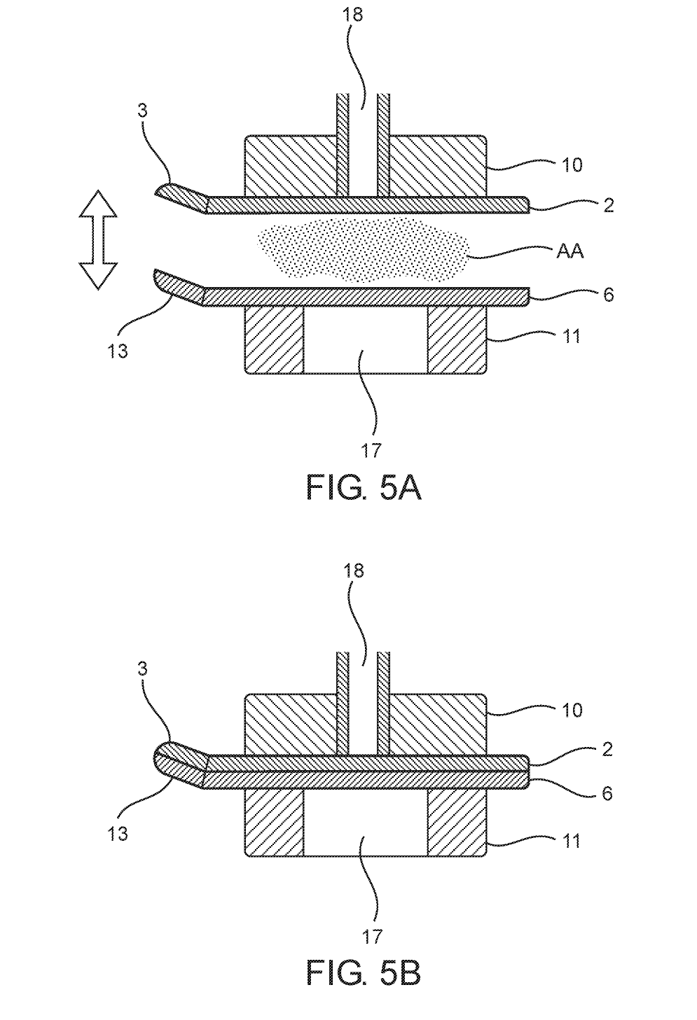

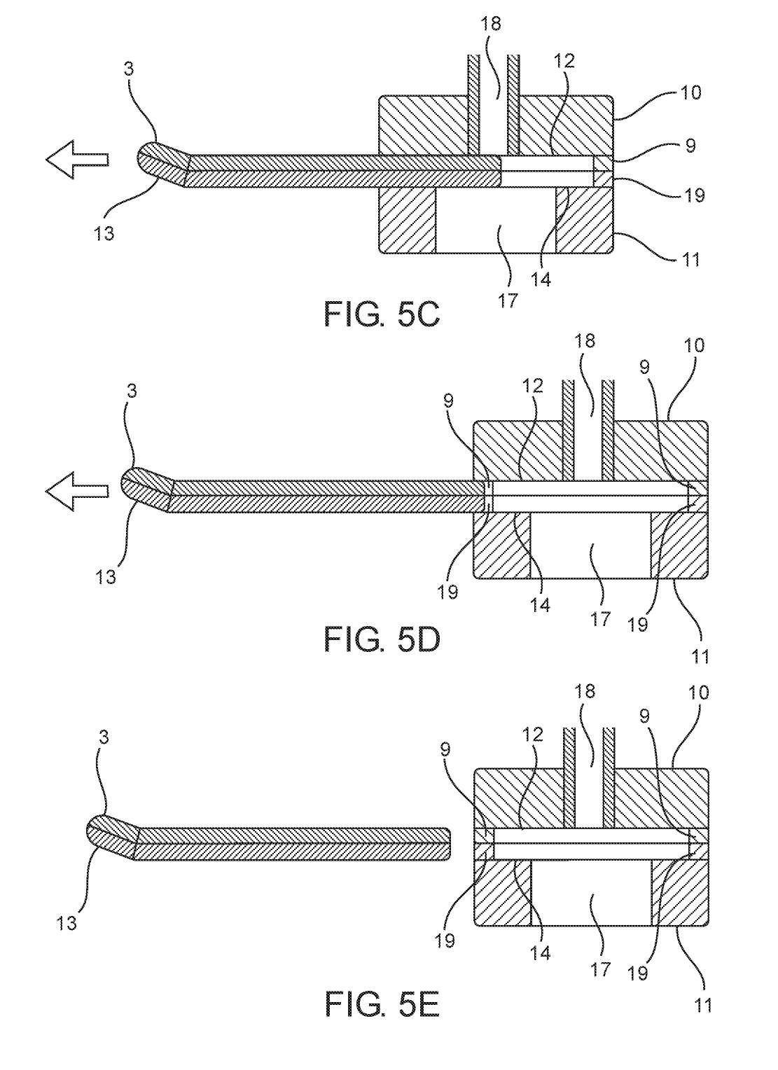

FIGS. 5A-5E present connection interfaces 2 and 6 of decontamination system 1 which further includes a first rail mechanism 12 configured to provide external displacement of first connection interface 2 and a second rail mechanism 14 configured to provide external displacement of second connection interface 6 (shown in FIGS. 5C-5E). FIG. 5A presents ports 10 and 11 of vessels 4 and 8, sealed by the connection interfaces 2 and 6, respectively. A seal (not shown) or cover (not shown) may be disposed between port 10 and first connection interface 2 which keeps the fluid passageway 18 of vessel 4 sealed during and/or after external displacement of connection interface 2. A seal (not shown) or cover (not shown) may be disposed between port 11 and second connection interface 6 which keeps the fluid passageway 17 of vessel 8 sealed during and/or after external displacement of connection interface 6. The seal (not shown) or cover (not shown) may be frangible and/or pierceable. FIG. 5B presents an engagement between the interfaces 2 and 6 and ports 10 and 11. FIGS. 5C and 5D show the ports 10 and 11 during the external displacement of connection interfaces 2 and 6. Following engagement of the interfaces, the interfaces 2 and 6 are externally displaced, optionally by sliding axially the interfaces along rails 12 and 14. Aperture 9 is resealed at about the time, prior to or thereafter the external displacement, optionally by a valve (not shown). FIG. 5E shows the ports 10 and 11 after the external displacement. Aperture 19 is resealed at about the time, prior to or thereafter the external displacement, optionally by a valve (not shown). Apertures 9 and 19 are configured to displace connection interfaces 2 and 6 therethrough while maintaining the hermetic seal between the first vessel 4 and the second vessel 8. The ports are securely engaged, and the openings 18 and 17 maintain fluid communication between the vessels 4 and 8. The interfaces may be displaced externally by pulling out tabs 3 and 13. Accordingly, first connection interface 2 and second connection interface 6 are configured to slide with respect to first vessel 4 and second vessel 8.

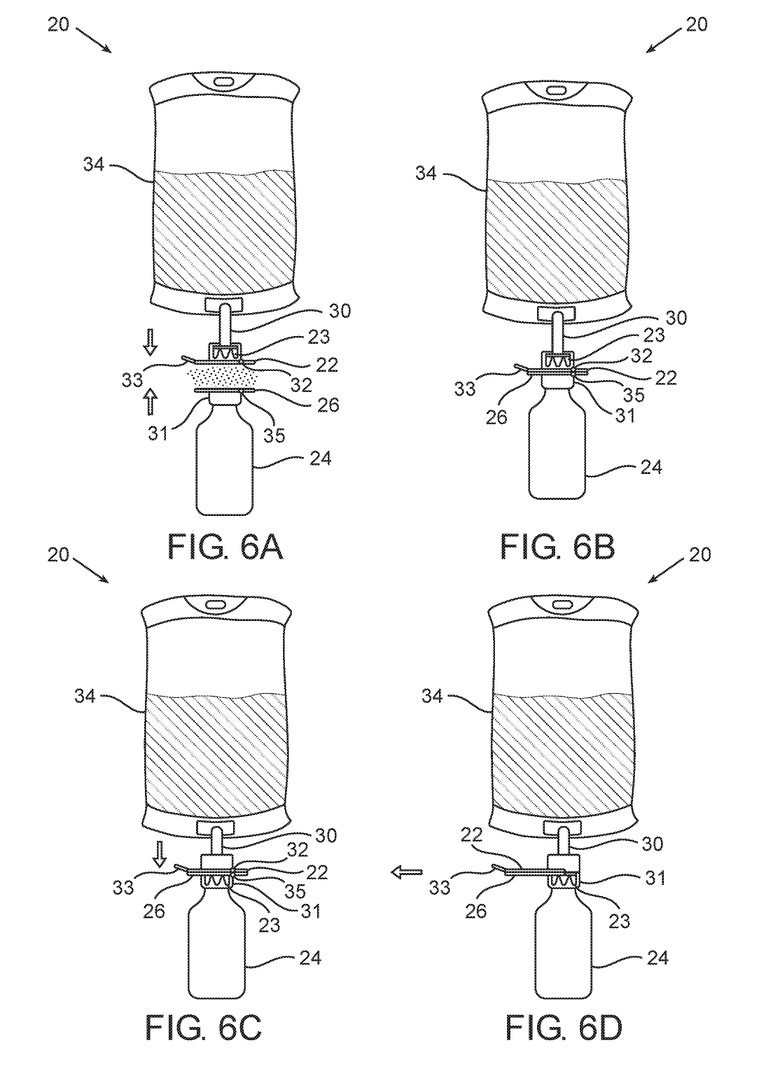

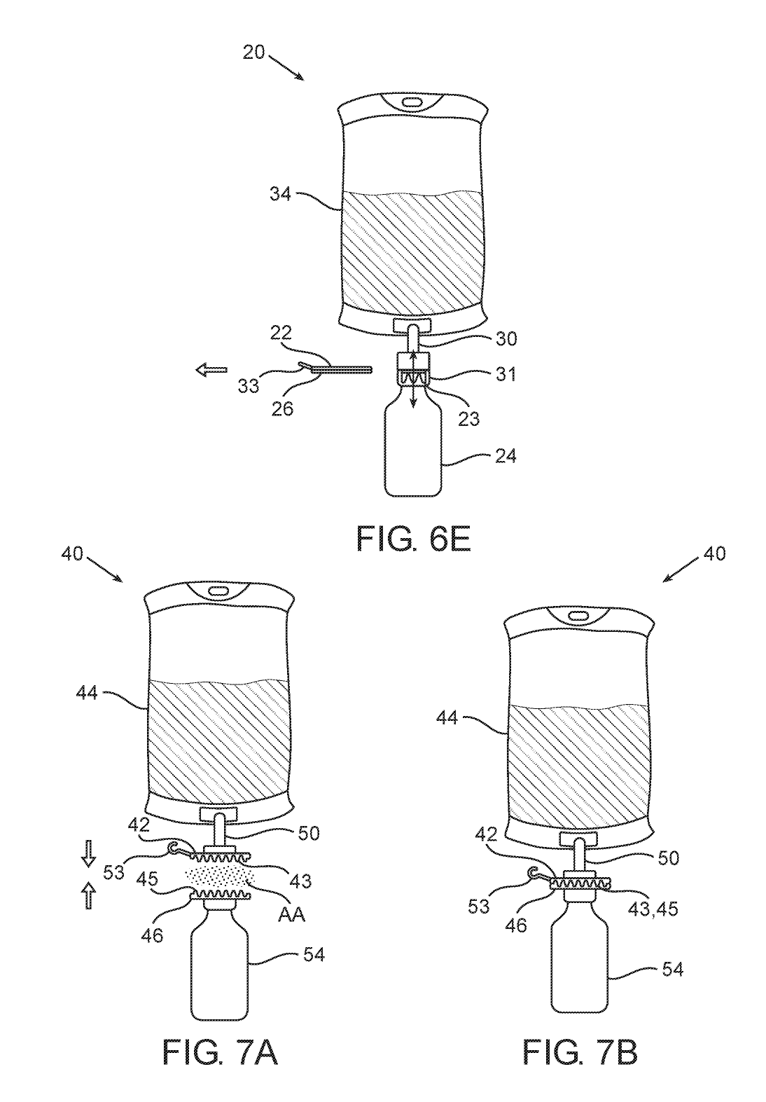

Reference is now made to FIGS. 6A-6E which illustrate a further exemplary decontamination system 20 of the invention. Decontamination system 20 includes a first connection interface 22 attached to a first vessel 34, optionally, via a port 30. Decontamination system 20 further includes a second connection interface 26 attached to a second vessel 24, optionally, via a port 31. Decontamination system 20 further comprises an engagement mechanism 23 configured to provide a secure engagement between first vessel 34 and second vessel 24. Engagement mechanism 23 is shown when located on port 30 of the first vessel 34. Nevertheless, engagement mechanism 23 may be positioned also on the second vessel 24, or on both vessels 24 and 34. Optionally, engagement mechanism 23 may abut a wall of the first vessel 34 and/or of the second vessel 24. Engagement mechanism 23 includes ratchet teeth mechanism, but other forms of an engagement mechanism are contemplated, such as a thread, a luer, and an adhesive tape. System 20 further includes lock-unlock mechanism 32 and 35 located on first connection interface 22 and second connection interface 26, respectively. Lock-unlock mechanism 32 and 35 are configured to allow locking and/or engagement of the connection interfaces, such that first connection interface 22 and second connection interface 26 are locked to each other at about the time of engagement thereof or after vessel engagement and are unlocked from any previous connection to ports 30 and 31.

It is to be noted that lock-unlock mechanism 32 and 35 may be disposed onto one or both vessels, vessel's ports, or connection interfaces. Lock-unlock mechanism 32 and 35 may additionally, allow locking the connection interfaces 22 and 26 to their respective vessel and unlock thereof at about the time or following engagement of the vessels 34 and 24 and/or connection interfaces 22 and 26 to allow the external displacement.

FIG. 6A shows the first and second connection interfaces 22 and 26, when spaced apart and when ambient air AA particles interfacing therebetween. FIG. 6B shows the interfaces when attached to each other and wherein AA particles entrapped between the interfaces. FIG. 6C shows the engagement mechanism 23 which allows a secure engagement between first vessel 34 and second vessel 24. The engagement mechanism 23 may be twisted and/or pushed downwardly and engage with a complementary thread or teeth or any other mechanism located on second vessel 24.

FIG. 6D shows the connection interfaces 22 and 26 when externally displaced from the vessels 34 and 24, respectively. Tab 33 allows for an external displacement of first connection interface 22 and optionally also of second connection interface 26. Tab 33 can be pulled out axially by a user, allowing for a displacement of the connection interface 22 and 26 from the first and second vessels 34 and 24, respectively. FIG. 6E shows decontamination system 20 after external displacement of the connection interfaces. The decontamination system 20 establishes a hermetically sealed connection of the two vessels which is maintained during external displacement of the connection interfaces and while the vessels remain in contact. The engagement between the first and second vessels 34, and 24, respectively, results in an ambient air AA particle-free engagement, wherein air particles interfacing the first and second interfaces are entrapped by the interfaces. In an embodiment of the invention, the first and second vessels 34 and 24, respectively, are in fluid communication with one another after external displacement of the connection interfaces 22 and 26 from first and second vessels 34 and 24, receptively.

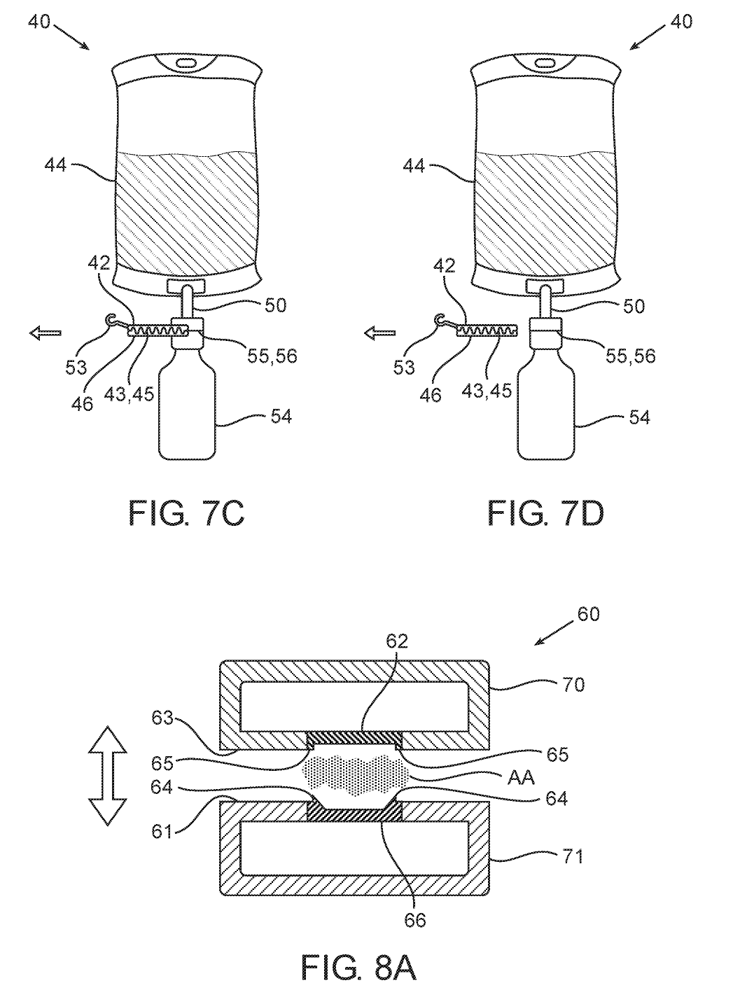

Reference is now made to FIGS. 7A-7D which show a further exemplary decontamination system 40 of the invention. The system 40 includes a first connection interface 42 attached to first vessel 44 and a second connection interface 46 attached to second vessel 54. The first connection interface 42 may be coupled to, optionally, integrally manufactured with, first vessel 44. The second connection interface 46 may be coupled to, optionally, integrally manufactured with, second vessel 54.

The connection interfaces 42 and 46 further comprise an engagement mechanism in the form of ratchet teeth 43 and 45 positioned on interfaces 42 and 46, respectively. Engagement mechanism 43/45 is configured to provide a secure engagement between the interfaces and to form an airtight seal between the first connection interface and the second connection interface. Other engagement mechanisms that may secure the first connection interface 42 to a second connection interface 46 are contemplated and may include an adhesive that secures the first connection interface 42 to the second connection interface 46 or an indentation that may secure the connection interfaces. The engagement mechanism 43/45 includes a mechanical object in the form of a hook 53. Hook 53 allows for a simultaneous external displacement of first and second connection interfaces 42 and 45. Optionally, the hook 53 may be a handle or a tab.

FIG. 7A shows the first and second connection interfaces 42 and 46, when spaced apart and when ambient air AA particles interfacing therebetween the interfaces. FIG. 7B shows the interfaces when attached to each other via the engagement mechanism 43 and 45 which interlocks the interfaces and allows to entrap the AA between the interfaces 42 and 46.

FIG. 7C shows the connection interfaces 42 and 46 while externally displacing from the vessels 44 and 54, respectively. Hook 53 allows the user to externally displace first connection interface 42 and second connection interface 46. Hook 53 can be pulled axially by a user, allowing for a displacement of the connection interfaces 42 and 46. Decontamination system 40 may further comprise a first rail mechanism 55 on first vessel 44 that is configured to provide external displacement of the first connection interface 42 via a sliding motion. A second rail mechanism 56 configured to provide external displacement of second connection interface 46 via a sliding motion, may be positioned on second vessel 54. The rail mechanism 55 may be positioned on a port 50 of vessel 44. Optionally, rail mechanism 55 may abut a wall, may be flush mounted or may be surface mounted to a wall of vessel 44. FIG. 7D shows decontamination system 40 after external displacement of the connection interfaces. The decontamination system 40 establishes a hermetically sealed and decontaminated connection of the two vessels which is maintained as long as the connection interfaces are pulled out and while the vessels remain in contact. The engagement between the vessels 44 and 54 results in an ambient air AA particle-free engagement and allows fluid communication therebetween the vessels. In an embodiment of the invention, when first connection interface 42 or second connection interface 46 slides off the first 55 and/or second rail 56 mechanisms, the hermetic connection between the first 44 and second vessels 54 is maintained. In an embodiment of the invention, the first connection interface 42 and the second connection interface 46 externally displace from the engagement between the first 44 and second 54 vessels at or about the same time.

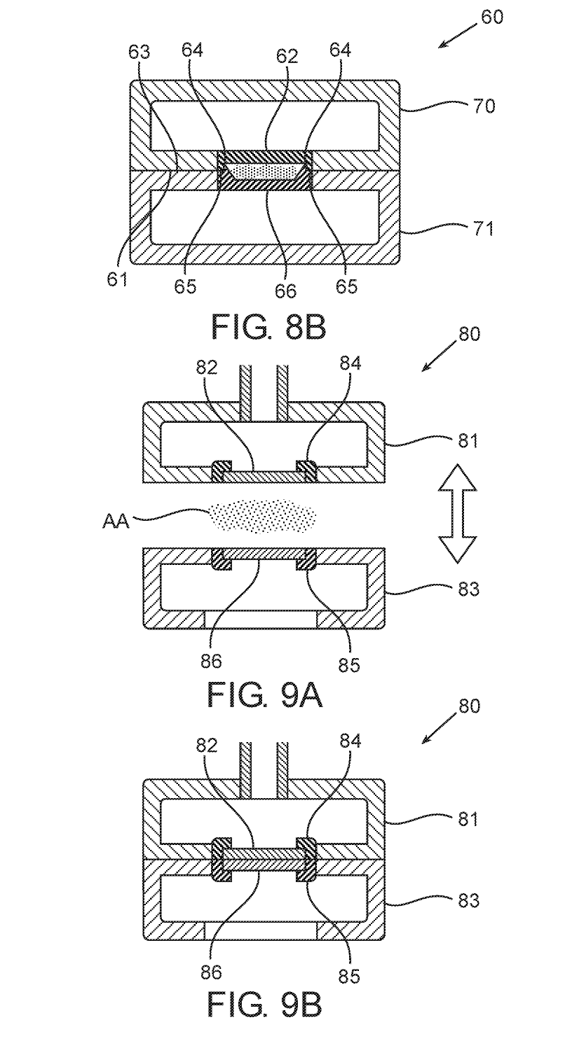

Reference is now made to FIGS. 8A-8B demonstrating decontamination system 60 comprising a first connection interface 62 attached to port 70 of a first vessel (not shown) and a second connection interface 66 attached to port 71 of a second vessel (not shown). First connection interface 62 may include opposing longitudinal ends 65. Second connection interface 66 may include opposing longitudinal ends 64. Ends 65 are entirely concealed within the hermetically sealed connection between the first vessel and the second vessel (shown in FIG. 8B). Ends 64 are an exterior portion of the connection interface 66 and protrude from upper wall 61 of port 71. Accordingly, ends 64 are exposed to contaminants. Ends 65 do not protrude from lower wall 63 of port 70 and their portions facing wall 63 are not exposed to contaminants. Accordingly, upon engagement of first connection interface 62 and second connection interface 66 ambient air particles are entrapped between the first 62 and second 66 connection interfaces (shown in FIG. 8B)

Reference is now made to FIGS. 9A-9B that illustrate back cut views of yet a further exemplary system 80 that includes a first connection interface 82 coupled to first vessel 81 and second connection interface 86 coupled to second vessel 83. First rail mechanism 84 is configured to allow the external displacement of first connection interface 82 and second rail mechanism 85 is configured to provide the external displacement of second connection interface 86. The exterior surfaces of the first connection interface 82 and the second connection interface 86 are about equal in surface area. First connection interface 82 may remain attached to first rail mechanism 84 or to first vessel 81 by a frangible seal or an adhesive. Upon external displacement of first connection interface 82 from first rail mechanism 84 or from first vessel 81 the frangible seal or the adhesive shall break or release the first connection interface 82 from the sealed position on the first rail mechanism 84 or from the first vessel 81. Alternately, first and second connection interfaces 82 and 86 may remain attached to first and second rail mechanisms 84 and 85 or to first and second vessels 81 and 83 respectively by a frangible seal or an adhesive. Upon external displacement of first and second connection interfaces 82 and 86 from first and second rail mechanisms 84 and 85 or from first and second vessels 81 and 83 the frangible seal or the adhesive shall break or release the first and second connection interfaces 82 and 86 from the sealed position on the first and second rail mechanisms 84 and 85 or from the first and second vessels 81 and 83.

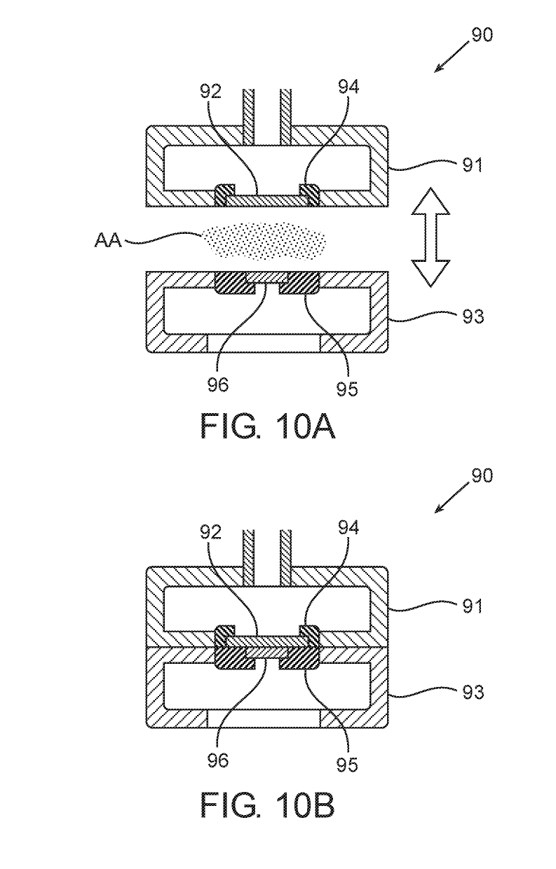

Reference is now made to FIGS. 10A-10B that illustrate back cut views of yet a further exemplary system 90 that includes a first connection interface 92 coupled to first vessel 91 and second connection interface 96 coupled to second vessel 93. First rail mechanism 94 is configured to allow the external displacement of first connection interface 92 and a second rail mechanism 95 is configured to allow the external displacement of second connection interface 96. The exterior surfaces of the first connection interface 92 and the second connection interface 96 are not equal. In one or more embodiments, the difference in surface area between the exterior surface area of the first connection interface 92 and the exterior surface area of the second connection interface 96 is less than 0.3 inches squared. In one or more embodiments, a portion (not shown) of first rail mechanism 94 may extend over an external portion (facing ambient air particles/contaminants AA) of first connection interface 92 thus preventing first connection interface 92 from falling off of first rail mechanism 94. In one or more embodiments, a portion (not shown) of second rail mechanism 95 may extend over an external portion (facing ambient air particles/contaminants AA) of second connection interface 96 thus preventing second connection interface 96 from falling off of second rail mechanism 95.

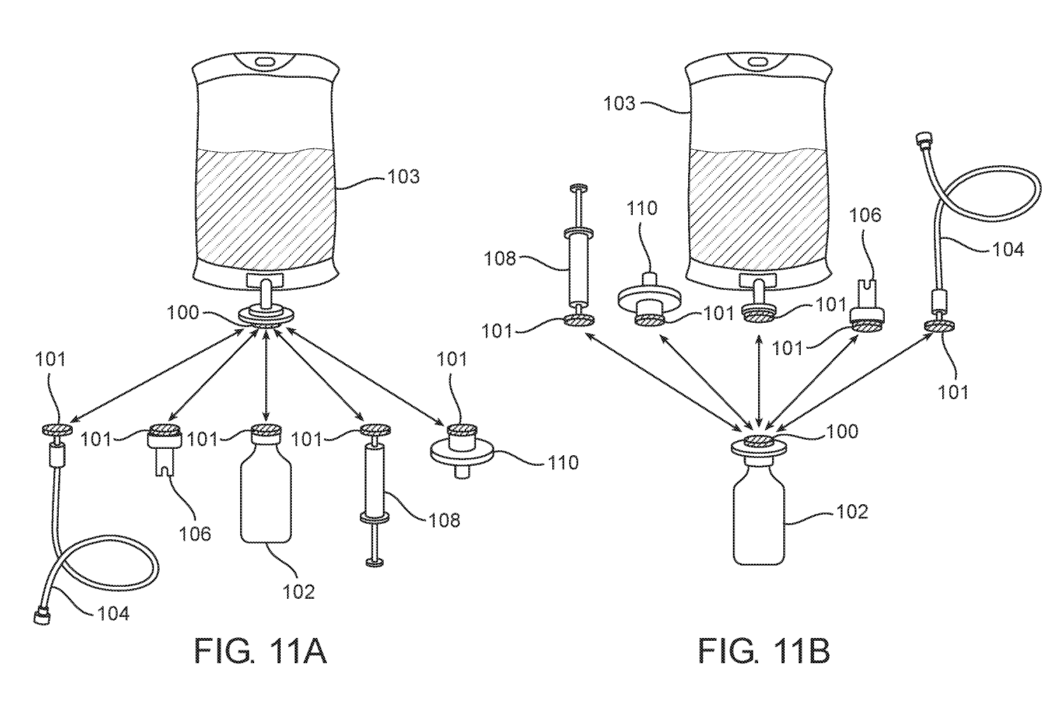

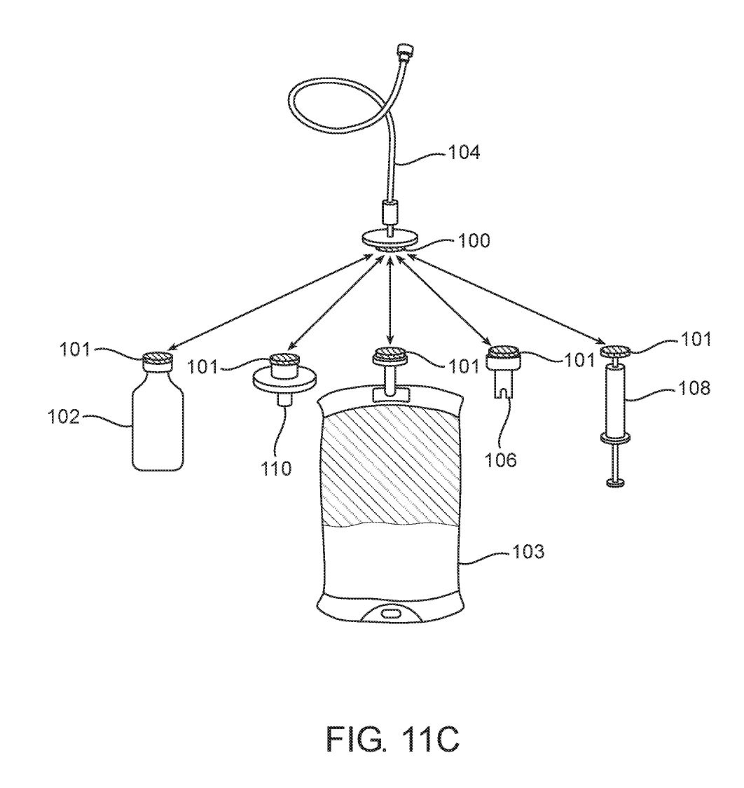

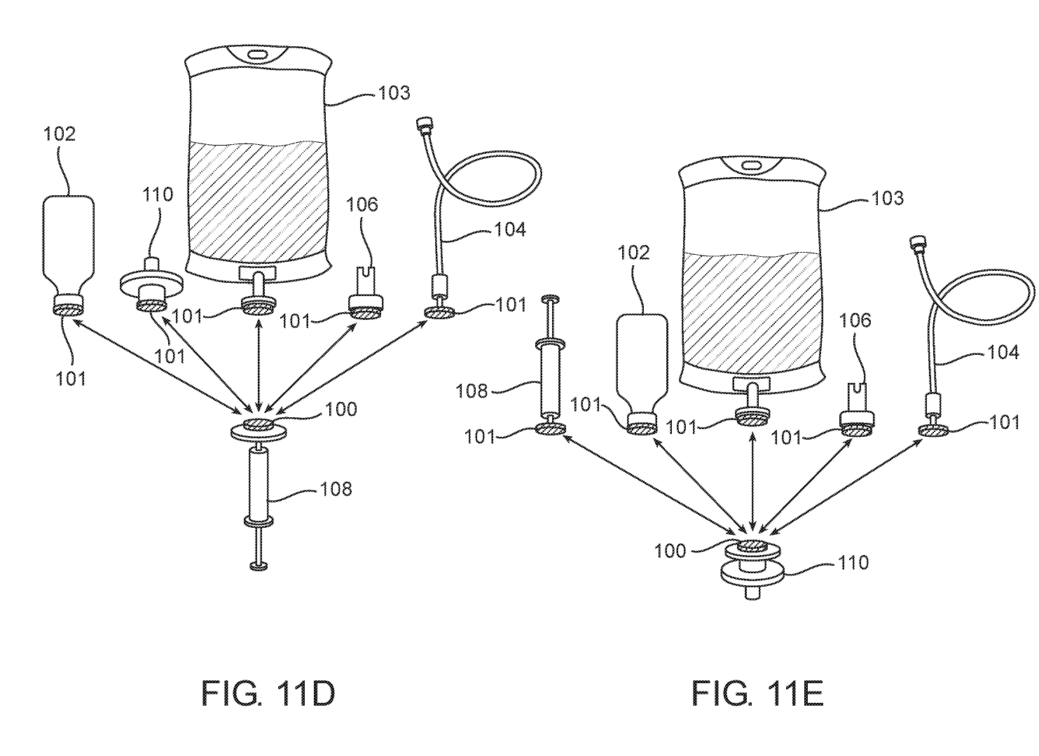

Reference is now made to FIGS. 11A-11E that illustrate connection interface 100 that may be attached to, optionally integrally manufactured with, a vessel, which may be a bag 103 (shown in FIG. 11A), a vial 102 (shown in FIG. 11B), an infusion line 104 (shown in FIG. 11C), a syringe 108 (shown in FIG. 11D), or a filter 110 (shown in FIG. 11E). A second vessel, such as vial 102, bag 103, infusion line 104, connector 106, syringe 108, or filter 110 can attach to connection interface 100 via connection interface 101.

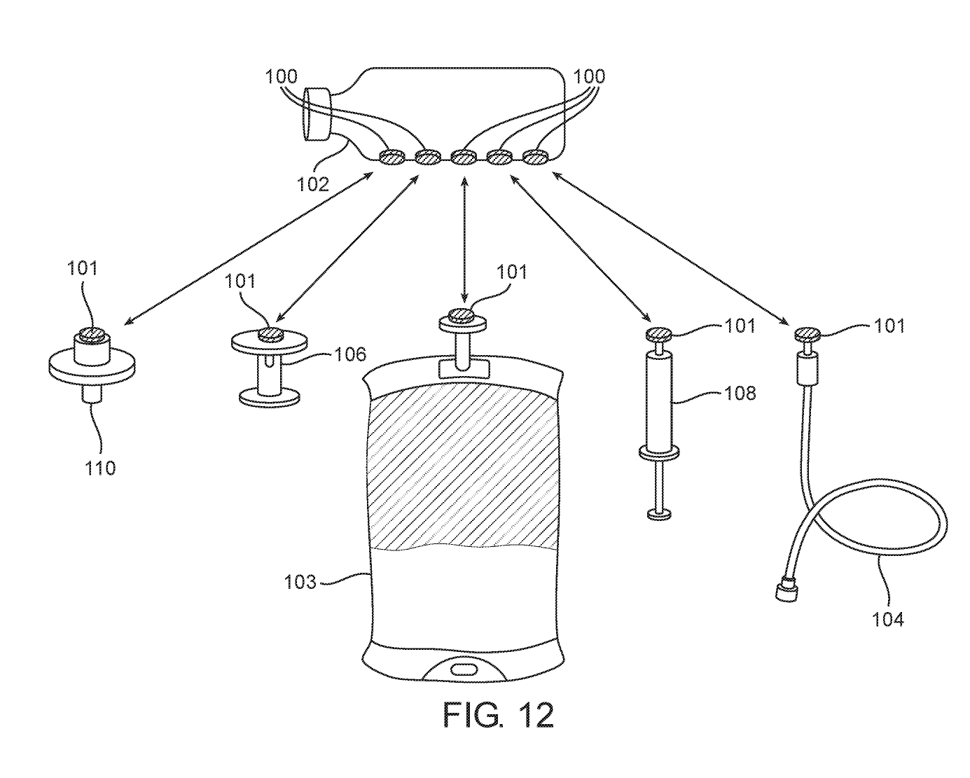

FIG. 12 schematically illustrates a vessel which is vial 102 coupled to a plurality of connection interfaces 100 which can be coupled to connection interface 101 which is attached to infusion line 104, syringe 108, bag 103, connector 106, and/or filter 110. The vessel 102 may also be a bag, a bottle or a syringe having a plurality of connection interfaces 100. The plurality of connection interfaces 100 may be externally displaceable connection interfaces. The vessel 102 may also have a plurality of rail mechanisms (not shown) configured to provide for the external displacement of the plurality of connection interfaces 100. The plurality of connection interfaces 100 of vessel 102 may abut a wall of vessel 102, may be surface mounted to vessel 102 or may be flush mounted to vessel 102. Optionally, vessel 102 may have a plurality of hinge mechanisms (not shown) configured to provide for the external displacement of the plurality of connection interfaces 100 of vessel 102. Optionally vessels/devices 103, 104, 106, 108 and 110 may have hinge mechanisms (not shown) configured to provide for the external displacement of connection interfaces 101 from their respective vessels/device. Optionally, vessel 102 may have a plurality of connection interfaces 100 that are peelable from vessel 102 and that are configured to engage with a plurality of peelable connection interfaces 101 of vessels/devices 103, 104, 106, 108 and 110.

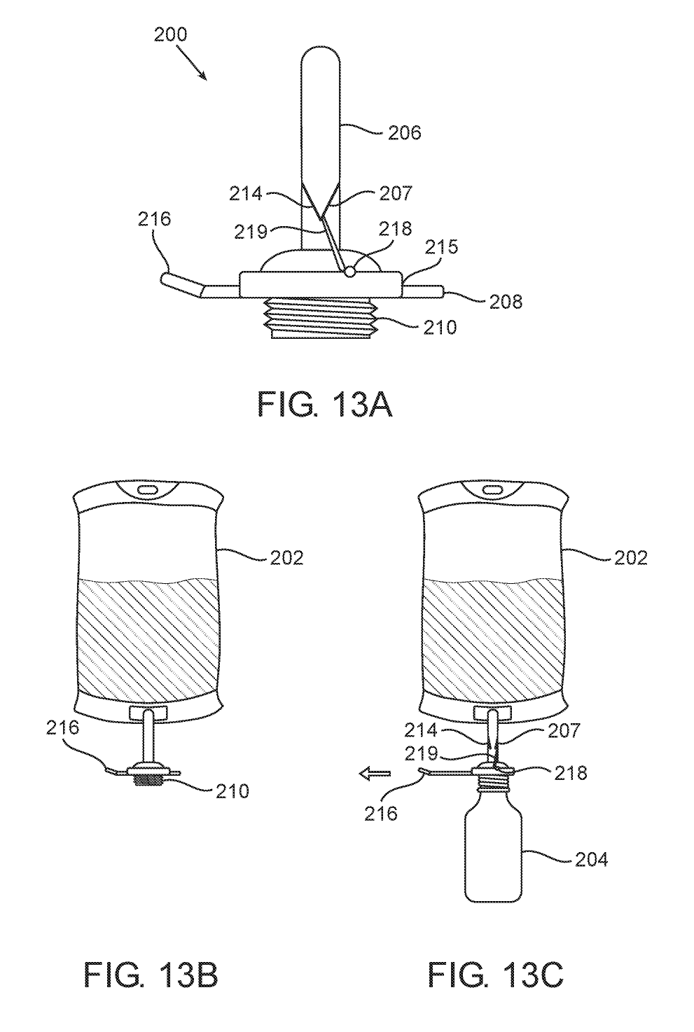

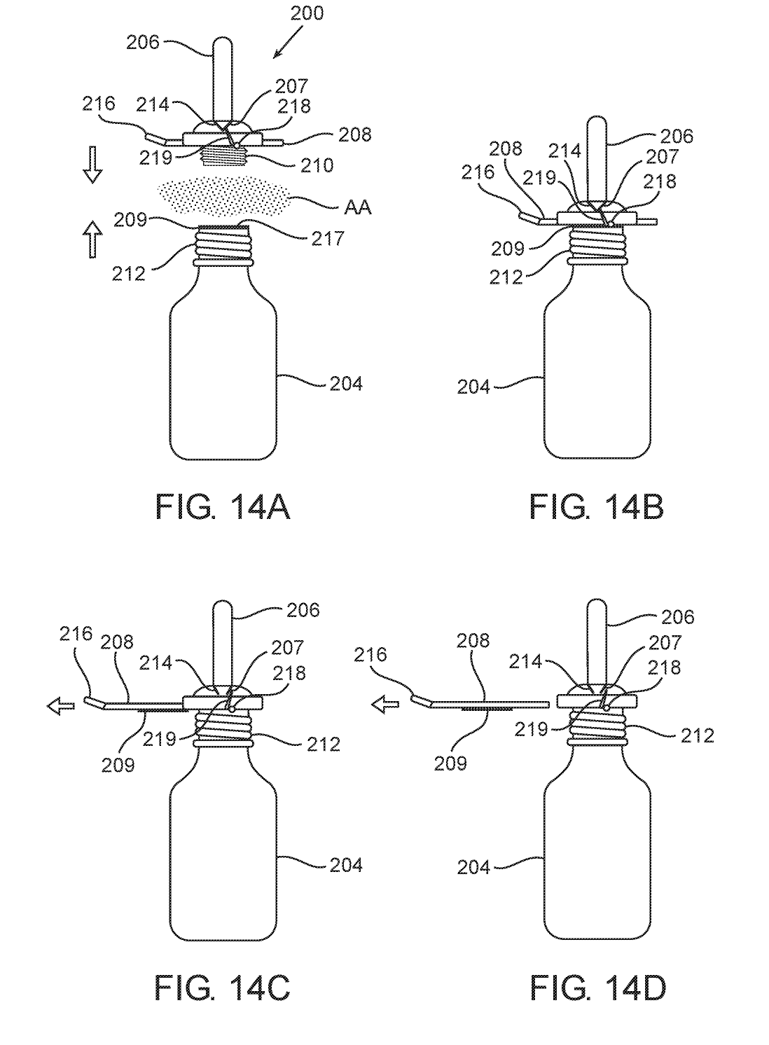

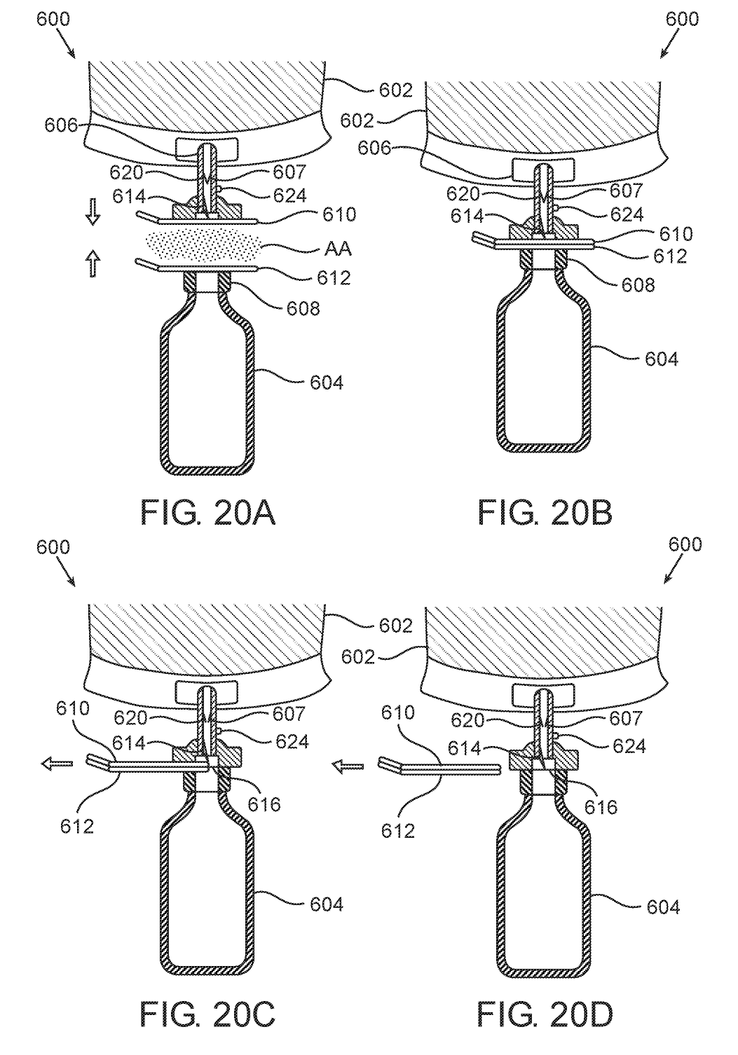

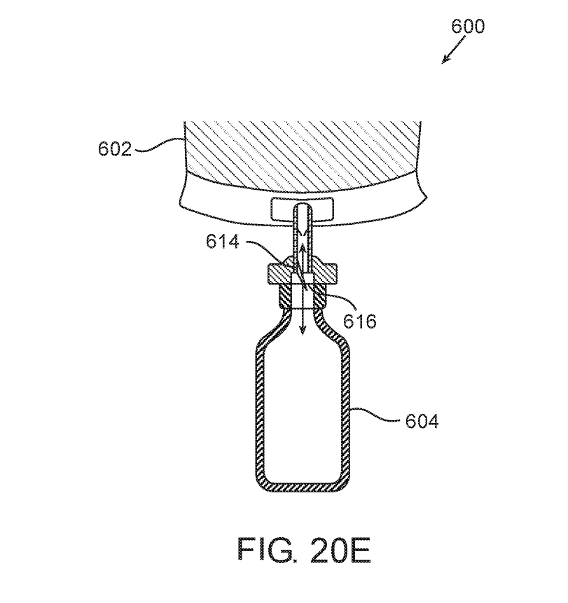

FIGS. 13A-13C shows an exemplary decontamination connector 200 configured for decontaminating a connection between vessels 202 and 204. The decontamination connector 200 includes a port 206 that can be attached to first vessel 202. The decontamination connector 200 further includes a connection interface 208 that is configured to cover port 206 of the decontamination connector 200. Connection interface 208 may be coupled to port 206. Connection interface 208 may be integrally manufactured with port 206 or may be manufactured separately and connected thereto subsequently in a sterile manner. Connection interface 208 and/or port 206 may be attached to first vessel 202. First vessel 202 may be a container, device or medical device. Connection interface 208 is configured to displace externally from port 206 of the decontamination connector 200. Decontamination connector 200 further includes an engagement mechanism 210 in the form of a thread, although alternative forms of engagement mechanisms are contemplated. The engagement mechanism 210 is configured to provide coupling between port 206 and second vessel 204. The engagement mechanism 210 further provides a secure engagement between decontamination connector 200 and second vessel 204. Valves 207 and 214 may be disposed within port 206, or within port 212 (shown in FIG. 14A) of vessel 204. The valves 207 and 214 are configured to be closed prior to engagement and external displacement of the connection interface 208 (shown in FIG. 13). The valves 207 and 214 configured to be opened at about or at the time of engagement and/or external displacement of the connection interface 208 (shown in FIG. 13C). Opening of valves 207 and 214 may be actuated by a user pushing down on an actuator 624 (shown in FIGS. 20A-20E), or by a twisting/turning motion of vessels in relation to each other. Lock-unlocking mechanism 218 may be disposed onto ports 206 and/or 212 (shown in FIG. 14A) such to allow unlocking of connection interface 208 from port 206 and connection interface 209 from port 212 (shown in FIG. 14A) at about the time of external displacement. Lock-unlocking mechanism 218 may additionally or alternatively include a hook 219 which is movable and may release or allow opening of valves 207 and 214. The hook 219 may actuate the opening of valves 207 and 214. Valves 207 and 214 may be one-way valves.

A sealed aperture or valve (not shown) may be disposed between port 206 and connection interface 208 and is configured to allow for the accommodation and external displacement of interface 208. Optionally, a frangible seal (not shown) may be disposed between port 206 and connection interface 208. The frangible seal may be breakable or rupturable by applying a force by hand. Optionally, a pierceable seal (not shown) may be disposed between port 206 and connection interface 208. The pierceable seal may be pierced by a piercing member (not shown) of the connector or by a piercing member (not shown) of vessel 204. The decontamination connector 200 may further include a rail mechanism 215 positioned between port 206 and interface 208. Optionally, rail mechanism 215 may be positioned on the decontamination connector 200. The rail mechanism 215 is configured to provide for the external displacement, optionally via a sliding motion, of the connection interface 208. Alternatively, interface 208 may be peeled off when external displacement occurs. Interface 208 may be externally displaced via a pulling motion. Optionally, interface 208 may be peeled off via a pulling motion.

FIG. 13B shows connector 200 when attached or coupled to bag 202. The connector 200 may be integrally manufactured with bag 202 or may be manufactured separately from bag 202 and connected thereto subsequently in a sterile manner. The connector 200 may form a unitary structure with the bag 202. FIG. 13C shows engagement, via engagement mechanism 210 (shown FIB. 13B), between the connector 200 and second vessel 204. The engagement allows for a hermetically coupled connection between decontamination connector 200 and vessel 204. The connection interface 208 (shown FIG. 13A) is externally displaced from port 206 (shown FIG. 13A) while maintaining the hermetically coupled connection between vessel 202 and vessel 204. The decontamination connector 200 further includes a tab 216 configured to allow for the external displacement of the connection interface 208 with respect to port 206 of decontamination connector 200. Tab 216 is configured to be pulled axially by a user, the axial pulling of tab 216 is configured to displace connection interface 208 from port 206 while maintaining a hermetic seal between decontamination connector 200 and vessel 204. Valves 207 and 214 are opened at about the time and/or at the time of external displacement of connection interface 208 allowing fluid passageway between the vessels. While connection interface 208 externally displace, the valves 207 and 214 are activated to be open. Further lock-unlocking mechanism 218 unlocks any connection between connection interface 208 from port 206 allowing the external displacement of connection interface 208. Optionally, lock-unlocking mechanism 218 may be located on a wall, may abut a wall, may be surface mounted to a wall or may be flush mounted to a wall of vessel 202.

FIGS. 14A-14D show steps of connecting first vessel 202 and second vessel 204 in a decontaminated manner using decontamination connector 200. FIG. 14A shows decontamination connector 200 when spaced apart from second vessel 204. Connector 200 may attach to second connection interface 209 which may be coupled to, optionally integrally manufactured with second vessel 204. Connection interface 209 may be shorter in length than connection interface 208. Valves 207 and 214 are kept closed prior to engagement of vessels 202 and 204. FIG. 14B shows decontamination connector 200 when attached to second vessel 204, via second connection interface 209. Connection interface 209 may cover the entire top surface of port 212 of vessel 204. Optionally, connection interface 209 may partially cover the top surface of port 212 of vessel 204. Optionally, connection interface 209 may cover an aperture of vessel 204. Optionally, connection interface 209 may cover the entire aperture of vessel 204. Optionally, connection interface 209 may cover at least a portion of an aperture of vessel 204. The attachment can be mediated via adhesive tape 217 provided onto connection interface 209 or onto a surface of connection interface 208. The attachment between first connection interface 208 and second connection interface 209 allows to entrap the ambient air AA particles interfacing the connector 200 and vessel 204 and to form an airtight connection between the connector 200 or first vessel 202 and the second vessel 204. FIG. 14C shows external displacement of connection interfaces 208 and 209. Lock-unlocking mechanism 218 releases any connection between interfaces 208 and/or 209 and/or ports 206 and/or 212, respectively, allowing the external displacement of connection interfaces 208 and 209. Tab 216 allows for an external displacement of connection interfaces 208 and 209. Tab 216 can be pulled axially by a user, allowing for a displacement of the connection interfaces 208 and 209. Alternatively, or additionally, connection interface 208 and/or 209 can be externally displaced via a sliding motion. Valves 207 and 214 are open at about the time of external displacement. FIG. 14D shows decontamination connector 200 after external displacement of the connection interfaces 208 and 209. Connector 200 establishes a hermetically sealed and decontaminated connection of the vessels which is maintained when the connection interfaces are pulled out and while the vessels remain in contact. The engagement between the vessel 204 and the connector 200 results in an ambient air AA particle-free engagement and allows fluid communication therebetween two vessels. In an embodiment of the invention, when connection interface 208 displaces externally, the hermetic connection between the connector and/or first vessel and second vessel is maintained.

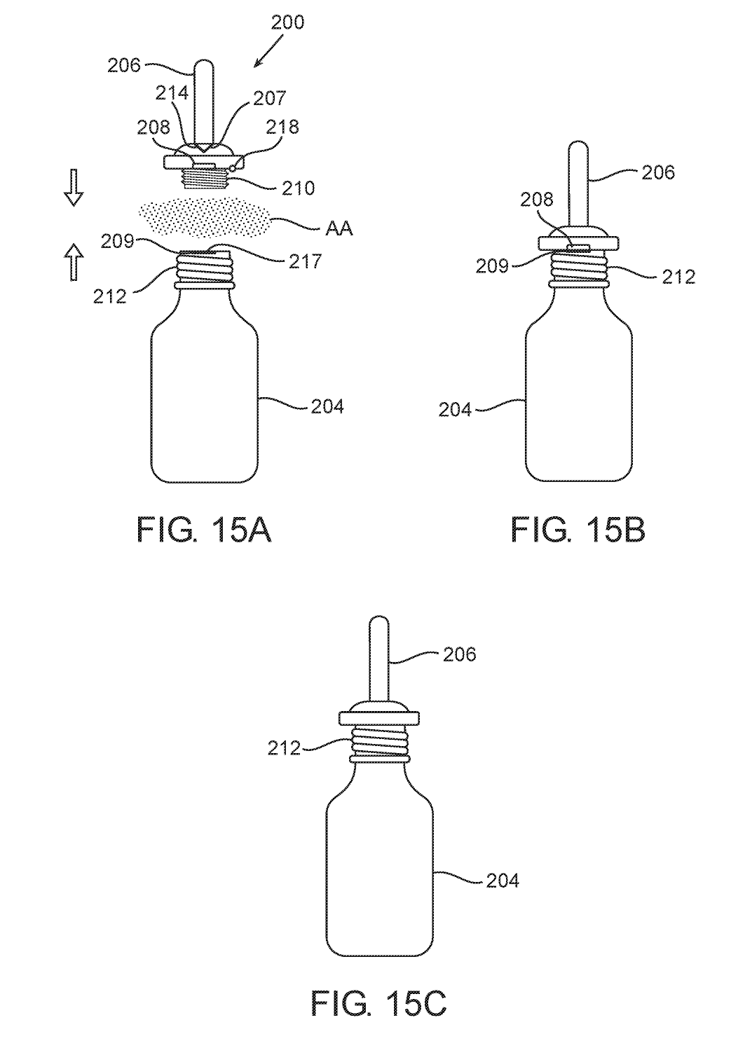

FIGS. 15A-15C are back views of connector 200. FIGS. 15A and 15B show the connection interface 208 that may be provided when submerged within port 206, optionally within aperture (not shown) disposed within port 206 and the connection interface 209 that may be provided when submerged within port 212, optionally within aperture (not shown) disposed within port 212. When the external displacement occurs, the apertures (not shown) disposed within ports 206 and 212 respectfully are resealed at during, about the time or prior to external displacement of connection interfaces 208 and 209, allowing for the hermetically sealed and air tight engagement of the vessels 202 and 204 at about the time or during external displacement of connection interfaces 208 and 209. FIG. 15C shows a resealed aperture after external displacement of connection interfaces 208 and 209.

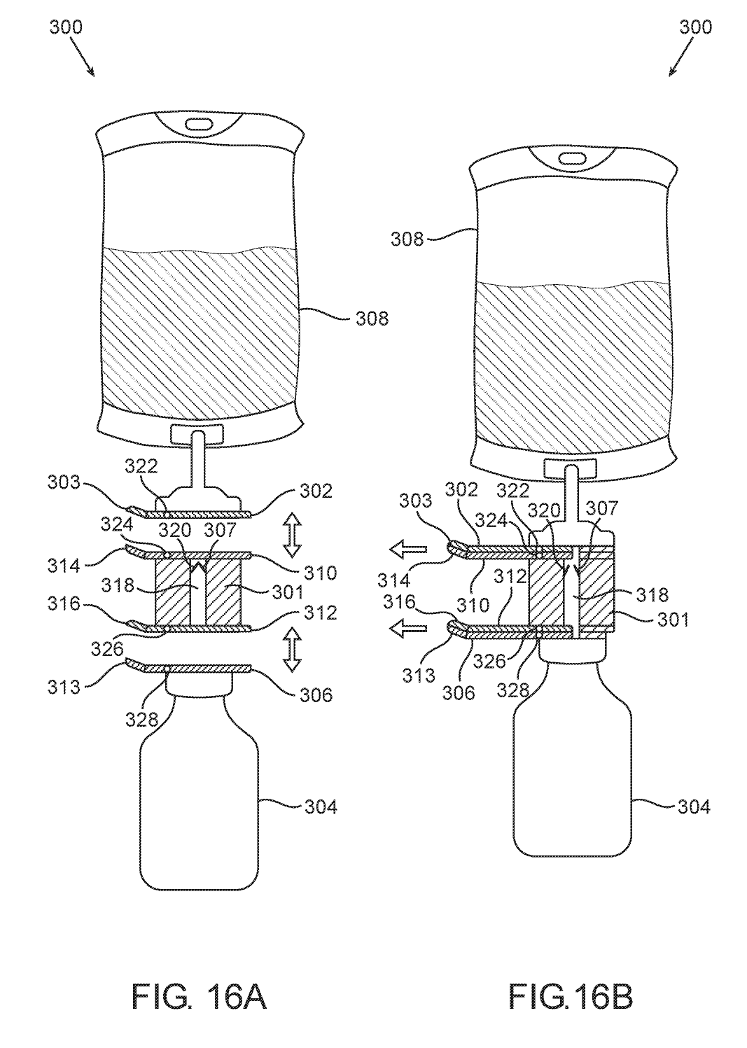

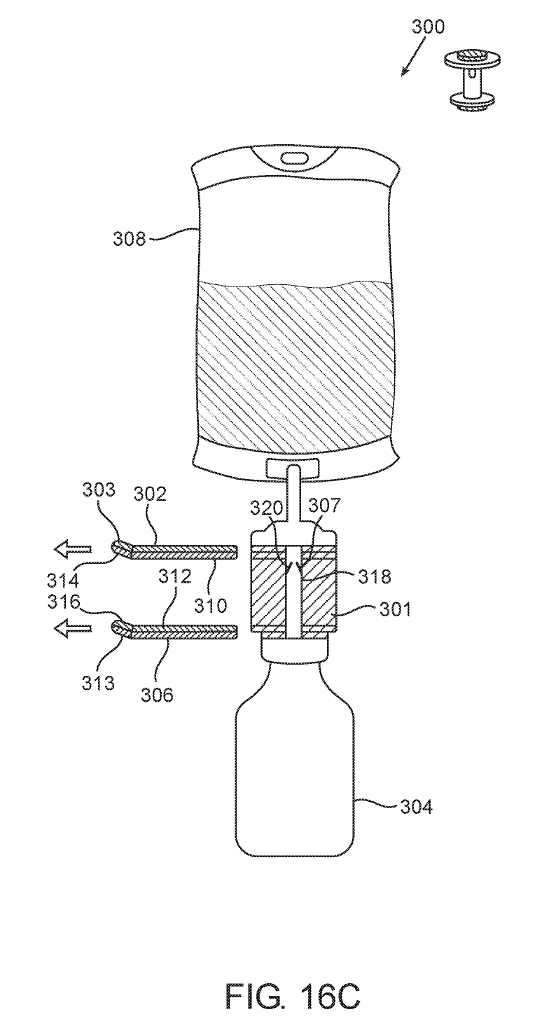

FIGS. 16A-16C are side cut views showing a system 300 that includes a first medical vessel 308 attached to a first connection interface 302 and a second medical vessel 304 attached to a second connection interface 306. A connector 301 which is coupled at opposition ends thereof to a third connection interface 310 and a fourth connection interface 312 is configured to allow the engagement of the vessels 308 and 304 and fluid passage way via a conduit 318 therethrough the vessels. System 300 may further include one or more valves 307 and 320 disposed within conduit 318 of connector 301. The valves are kept closed prior to the engagement between the vessels 304 and 308 and become open at about the time, during or after external displacement of the connection interfaces. In an embodiment of the invention, the valves 307 and 320 are opened during the external displacement. It is to be noted that valves 307 and 320 may be disposed onto alternative locations, for example, within vessels 304 and/or 308, and/or ports of said vessels. Lock-unlocking mechanism 322, 324, 326 and/or 328 may be disposed to lock the connection interfaces 302, 310, 312 and 306, respectively, prior to engagement of connection interfaces and vessels and unlock thereof at about the time of engagement, allowing the external displacement. The lock-unlock mechanism may allow one or more of: locking the connection interface to a vessel or a port thereof, locking connection interfaces to each other, unlocking connection interfaces to a vessel or a port thereof at the time of or prior to the external displacement, and controlling opening of valves located at the vessels ports. FIG. 16A depicts system 300 prior to engagement between the vessels 308 and 304. FIG. 16B depicts the connector 301 attached at opposing ends thereof to first vessel 308 and second vessel 304. First connection interface 302 is attached to third connection interface 310 and second connection interface 306 is attached to fourth connection interface 312. The connection interfaces can be externally displaced, for example, via a sliding motion by axially pulling at least one of tabs 303, and 314 and at least one of tabs 313 and 316. FIG. 16C depicts the engaged vessels 304 and 308 wherein a fluid passageway is provided following the external displacement of the connection interfaces via conduit 318. The connector 301 is maintained as long as the vessels are to be connected and used for the transfer of fluids. System 300 establishes a hermetically sealed and decontaminated connection between the two vessels 308 and 304 which is maintained during the external displacement of the connection interfaces and as long as the vessels remain in contact. The engagement, results in an AA particles-free engagement between the vessels. Ambient air AA interfacing the first and third connection interfaces 302 and 310, and the second and forth connection interfaces, 306 and 312, respectively, is entrapped by the engagement of the interfaces. Engagement between vessel 304 and connector 301 may occur via an engagement mechanism (not shown) on or attached to vessel 304 and/or on or attached to connector 301. The engagement mechanism may be selected from a thread, a luer, a ratchet teeth mechanism, a snap-on mechanism, a slide-on mechanism, an adhesive mechanism or combinations thereof. Engagement between vessel 308 and connector 301 may occur via an engagement mechanism (not shown) on or attached to vessel 308 and/or on or attached to connector 301. The engagement mechanism may be selected from a thread, a luer, a ratchet teeth mechanism, a snap-on mechanism, a slide-on mechanism, an adhesive mechanism or combinations thereof. The force of the engagement between connector 301 and vessel 304 maintains a hermetic connection between connector 301 and vessel 304 throughout or during external displacement of connection interfaces 306 and 312 and reseals the connection between connector 301 and vessel 304 prior to or at about the time of external displacement of connection interfaces 306 and 312. The force of engagement between connector 301 and vessel 308 maintains a hermetic connection between connector 301 and vessel 308 throughout or during external displacment of connection interfaces 302 and 310 and reseals the connection between connector 301 and vessel 308 prior to or at about the time of external displacement of connection interfaces 302 and 310.

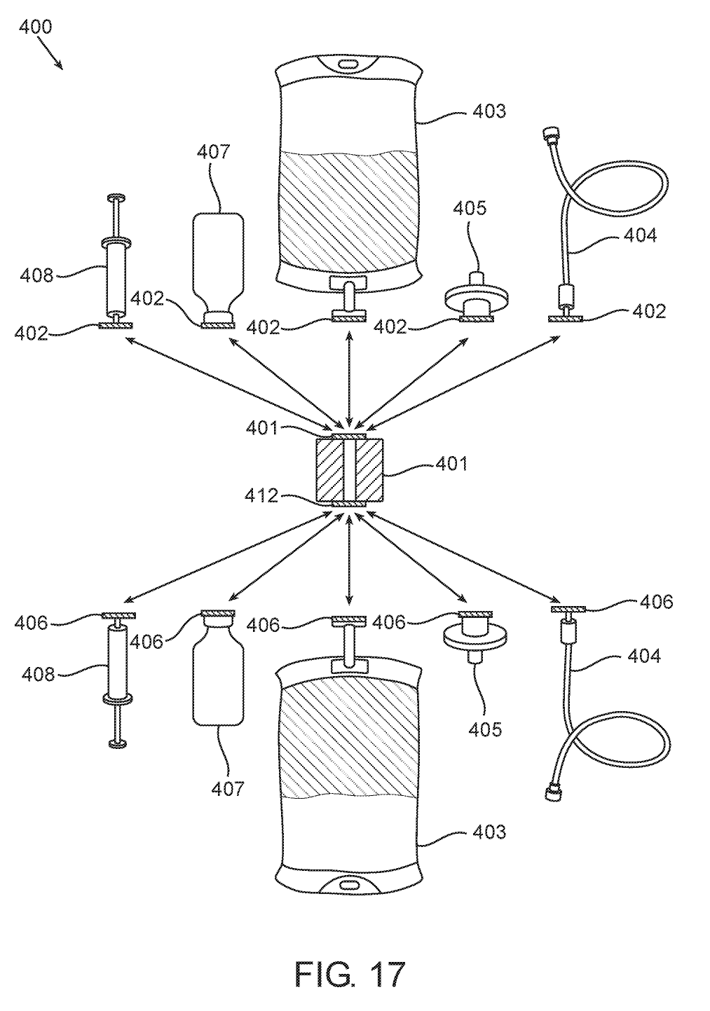

FIG. 17 depicts a system 400 which is similar to system 300 in that it includes a first connection interface 402 and a second connection interface 406 each of which may be coupled to a medical vessel of various types such as syringe 408, vial 407, bag 403, filter 405, or infusion line 404. The system 400 further includes a connector 401 attached at opposing ends thereof to a third connection interface 401 and a forth connection interface 412. First connection interface 402 may engage with third connection interface 401 and second connection interface 406 may engage with forth connection interface 412. The engagement of the interfaces allows to entrap air particles therebetween the interfaces. The interfaces can externally displace allowing a hermetic decontaminated engagement with fluid passageway between the engaged vessels. The connector 401 (positioned in the middle of FIG. 17 between the top and bottom row of vessels) may have a first rail mechanism (not shown) configured to provide for the external displacement of the third connection interface 401 and may have a second rail mechanism (not shown) configured to provide for the external displacement of the fourth connection interface 412. The first and second rail mechanisms (not shown) of the connector 401 may be positioned on or around the ends or openings of the conduit which is disposed within the connector 401. The third connection interface 401 may be disposed on and/or between the rails of the first rail mechanism located on the connector 401. The fourth connection interface 412 may be disposed on and/or between the rails of the second rail mechanism located on the connector 401. The connector 401 may further comprise at least one or a plurality of engagement mechanisms (not shown) or a retention member (not shown) configured to attach or retain at least one or a plurality of vessels. The engagement mechanism may be selected from but is not limited to a thread, a ratchet teeth mechanism, a snap-on mechanism, an adhesive or a slide-on mechanism. Optionally, the connector 401 may have more than two externally displaceable connection interfaces (not shown) so as to provide contaminant-free engagement between at least three vessels. Optionally, the connector 401 may have more than two rail mechanisms which provide for the external displacement of more than two connection interfaces. In accordance with this embodiment, the connector 401 may be a manifold. In one or more embodiments, the connector 401 may have at least two hinge mechanisms (not shown) that provide for the external displacement of at least two connection interfaces. In one or more embodiments, the connector 401 may have at least one, or a plurality, of peelable connection interfaces.

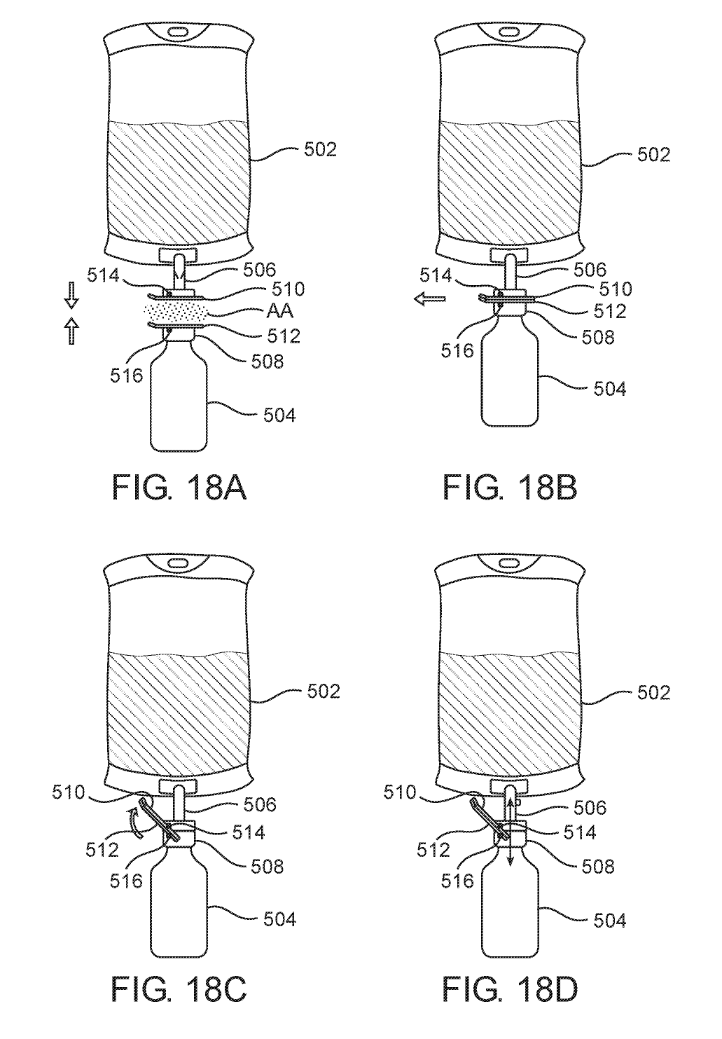

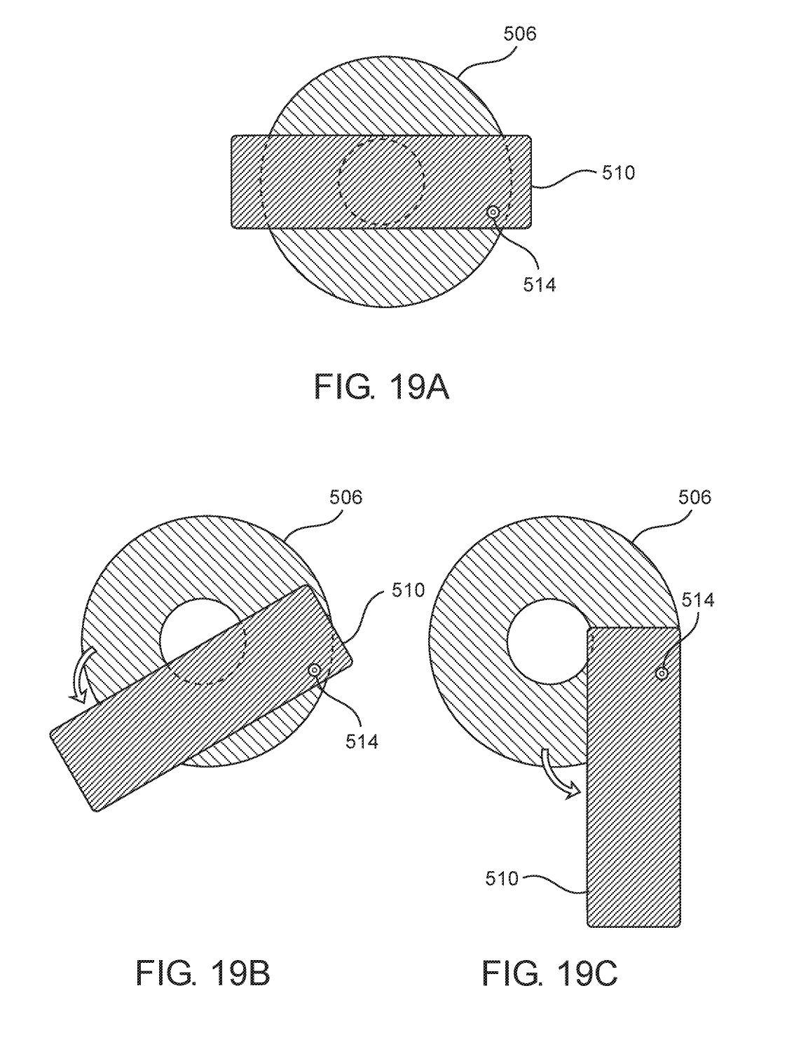

FIGS. 18A-18D denote yet a further exemplary system 500 that includes a first medical vessel 502 attached to a first connection interface 510 via port 506, and a second medical vessel 504 attached to a second connection interface 512 via port 508. Connection interface 510 is coupled to port 506 of first vessel 502 via a hinge mechanism 514. Connection interface 512 is coupled to port 508 of second vessel 504 via a hinge mechanism 516. FIG. 18A depicts system 500 prior to engagement between the vessels 502 and 504. FIG. 18B depicts system 500 wherein the interfaces 510 and 512 attached to each other. FIG. 18C shows the connection interfaces 510 and 512 while externally displacing from the vessels 502 and 504. The external displacement is mediated by an axial motion via the hinges 514 and 516. The connection interfaces 510 and 512 remain attached to ports 506 and 508 of vessels 502 and 504, respectively, during the external displacement.

The connection interfaces 510 and 512 may remain attached to hinges 514 and 516, respectively. The connection interfaces 510 and 512 remain attached to ports 506 and 508 of vessels 502 and 504, respectively, during the external displacement. The connection interfaces 510 and 512 may firstly move sideways, optionally via a sliding motion conducted through a rail mechanism and thereafter rotates upwards (as shown in FIGS. 18C and 18D) or downwards. FIG. 18D demonstrates the fluid passageway formed after the external displacement. The decontamination system 500 establishes a hermetically sealed apertures and decontaminated connection of the two vessels which is maintained as long as the vessels remain in contact. The engagement between the vessels 502 and 504 results in an ambient air AA particle-free engagement and allows fluid communication therebetween the vessels. It is to be noted that the hinge mechanism may be relevant to any of the herein disclosed connectors and/or connection interfaces and/or decontamination systems.

Reference is now made to FIGS. 19A-19C that show top views of connection interface 510. The connection interface 510 externally displaces from its initial location shown in FIG. 19A to subsequent positions shown in FIGS. 19B and 19C, thereby allowing a fluid passageway between vessels. Connection interface 510 rotates sideways by a hinge mechanism 514 which also allows attachment to port 506. The external displacement in this exemplary embodiment is formed solely by the hinge mechanism. The port 506 may be made of an elastomeric material, a rubber material, a plastic material, or combinations thereof. The airtight/hermetic connection between ports 506 and 508 remains intact via a pressure/force of engagement provided by the engagement between vessels 502 and 504 (shown in FIG. 18) by an engagement mechanism (not shown). The engagement is not limited to but may be selected from a thread/luer mechanism, ratchet teeth mechanism, an adhesive mechanism, and combinations thereof. Port 506 is resealed at about the time and/or during the external displacement of the connection interface 510 via the hinge mechanism 514 due to the pressure/force of engagement provided by said engagement mechanisms (not shown).

Reference is now made to FIGS. 20A-20E which illustrate yet a further exemplary decontamination system 600. Decontamination system 600 includes a first connection interface 610 attached to a first vessel 602 and a second connection interface 612 attached to a second vessel 604. Decontamination system 600 further includes a piercing member 614 configured to allow for the fluid passageway during or following the external displacement of the interfaces 610 and 612. Piercing member 614 may be present on first and/or second vessel 602 and 604, respectively. Piercing member 614 may include any suitable element that is capable of piercing a vessel surface seal 616 present to seal an opening of at least one of vessels 602 and 604. In an exemplary embodiment, piercing member 614 includes one or more sharp edge(s). In one or more exemplary embodiments, piercing member 614 includes a needle, such as a hollow needle. The piercing member 614 may lock into a final position once piercing a surface of at least one vessel 602 and/or 604. Optionally, the piercing member 614 may be retractable thus returning to an initial position after piercing a surface of at least one vessel 602 and/or 604. Vessel surface seal 616 may be a layer, a stopper, a rubber bung or a membrane, or optionally a thin film made of various materials that can be punctured, such as a plastic or aluminum foil. Optionally, vessel surface seal 616 may be frangible. In one or more embodiments, vessel 602 may also have a surface seal (not shown that is pierceable and/or breakable by the piercing member 614 or by a force applied to vessel 602 and/or 604.

One or more of valves 607 and 620 may be disposed onto and/or inside port 606 such that the valves 607 and 620 are closed prior to and/or during the external displacement of the connection interfaces 610 and 612. It is to be noted that similar or other valves, such as valves 607 and 620 may be disposed onto vessel 604, or port 608 and restrict fluid passageway to the stage that follows the external displacement. The valve 607 and or 620 may be a one-way valve. Valves 607 and 620 may be actuated by valve unlocking mechanism 624 which may be operated externally by a user. Valve unlocking mechanism 624 may be actuated by any of a twisting, a turning, a pushing motion and combinations thereof. In one or more embodiments, valve unlocking mechanism 624 may be also operable to actuate the piercing member 614. In one or more embodiments, the valve unlocking mechanism 624 may be actuated by the external displacement of connection interface 610 or by the external displacement of both connection interfaces 610 and 612. In one or more embodiments, the valve 607 and 620 may prevent backflow of fluid from the first vessel 602 to second vessel 604. It is to be noted that the piercing member and/or the valve unlocking mechanism 624 may be relevant to any of the herein disclosed connectors and/or connection interfaces and/or decontamination systems.

FIG. 20A shows decontamination system 600 of the invention prior to engagement, wherein ambient air AA particles surround the connection interfaces 610 and 612. FIG. 20B shows decontamination system 600 following an engagement and an entrapment of ambient air AA particles between first and second connection interfaces 610 and 612, respectively. FIG. 20C shows decontamination system 600 at about the time and/or during external displacement of first and second connection interfaces 610 and 612, respectively. FIG. 20D shows decontamination system 600 following the external displacement. At FIG. 20E, the piercing member 614 is shown when actuated to pierce an opening sealed by vessel surface seal 616 present on either or both openings of vessels 602 and 604. Piercing member 614 may be actuated via various mechanisms. For example, piercing member 614 is actuated via a twisting or turning motion, for example, twisting of one or both of the vessels towards the other vessel. For example, piercing member 614 is actuated via a pushing motion of one or both of the vessels towards the other vessel. For example, piercing member 614 is actuated via a twist and push or push and twist actions. In yet a further exemplary embodiment, piercing member 614 is actuated by pressing and/or operating an external button located on one or more of the vessels, and/or by operating a hinge or a rail mechanism. The external button according to this embodiment may be attached to piercing member 614 and pressed downwardly to allow piercing member 614 to puncture vessel surface seal 616.

Following the external displacement of the connection interfaces 610 and 612, piercing member 614 is actuated to pierce vessel surface seal 616 and thereby establish a fluid communication between the vessels 602 and 604. Piercing member 614 is thus movable from a first position wherein the piercing member 614 is concealed from ambient air particles and disposed within one or both vessels 602 and 604, to a second position wherein the piercing member 614 is actuated to establish the fluid passageway. Piercing member 614 pierces vessel surface seal 616 at about the time and/or following a hermetic engagement between vessels 602 and 604 or at about the time and/or following a hermetic engagement between connection interfaces 610 and 610. In an embodiment of the invention, the external displacement releases the piercing member 614 and allows it to move from the first to the second position. Optionally, piercing member 614 pierces at least one vessel surface seal 616 of vessel 604 and/or a vessel surface seal (not shown) of vessel 602 following external displacement of connection interfaces 610 and 612. Piercing member 614 may be locked into an initial position by a safety mechanism (not shown) disposed on or within port 606 or on or within vessel 602. The safety mechanism (not shown) prevents premature movement/activation and/or piercing of the piercing member prior to a hermetic engagement between vessels 602 and 604. Optionally, the safety mechanism (not shown) may be released or unlocked at or at about the time of engagement of vessels 602 and 604, at or at about the time of engagement of connection interfaces 610 and 612, and/or at or at about the time of external displacement of connection interfaces 610 and 612. It is to be noted that the herein disclosed piercing member, mechanism of action of the piercing member and safety mechanism may be relevant to any of the herein disclosed connectors and/or connection interfaces and/or decontamination systems. In one or more embodiments, the vessel surface seal 616 on vessel 602 and/or vessel 604 may be a frangible seal configured to be ruptured or broken by applying a force by hand to vessel 602 and/or vessel 604 and/or port 606 thus pushing fluid from vessel 602 or vessel 604 through the vessel surface seal 616.

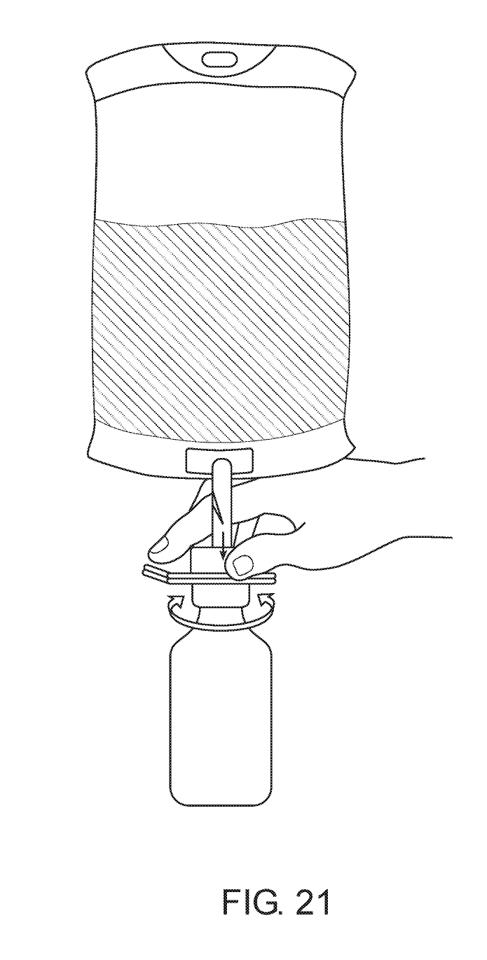

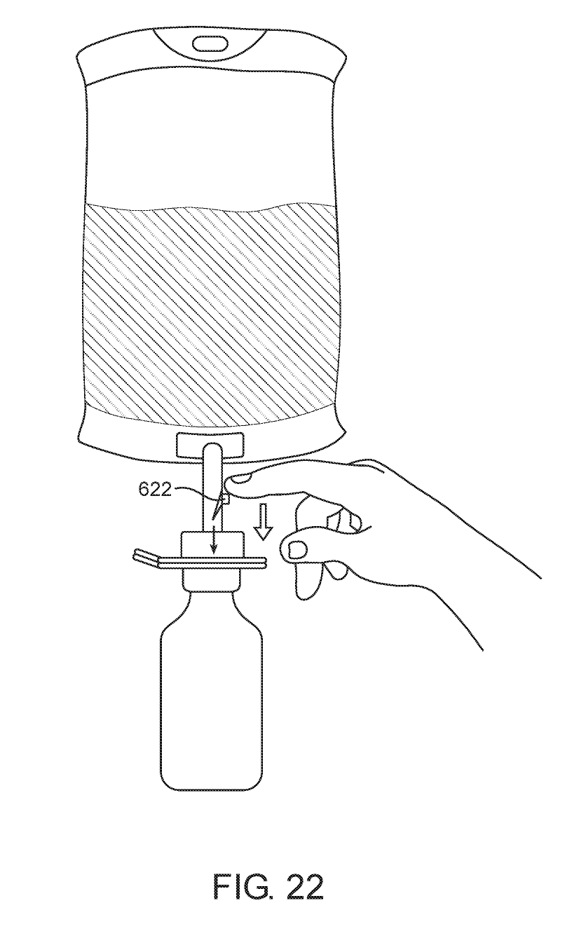

Reference is now made to FIG. 21 which is a schematic illustration presenting decontamination system 600 which includes a piercing member 614 that can be actuated by a twist motion. The twist motion may be clockwise or counterclockwise. In an alternative embodiment shown in FIG. 22, piercing member 614 can be actuated by a downward push motion of an actuator 622. Following an external displacement, a user pushes downwardly actuator 622 to allow piercing member 614 to puncture vessel surface seal 616 (FIG. 22). In one or more embodiments, when a piercing member is provided within the system or connector, the click sound, or another sound may alert the user that at least one interface has been pierced and fluid communication established.

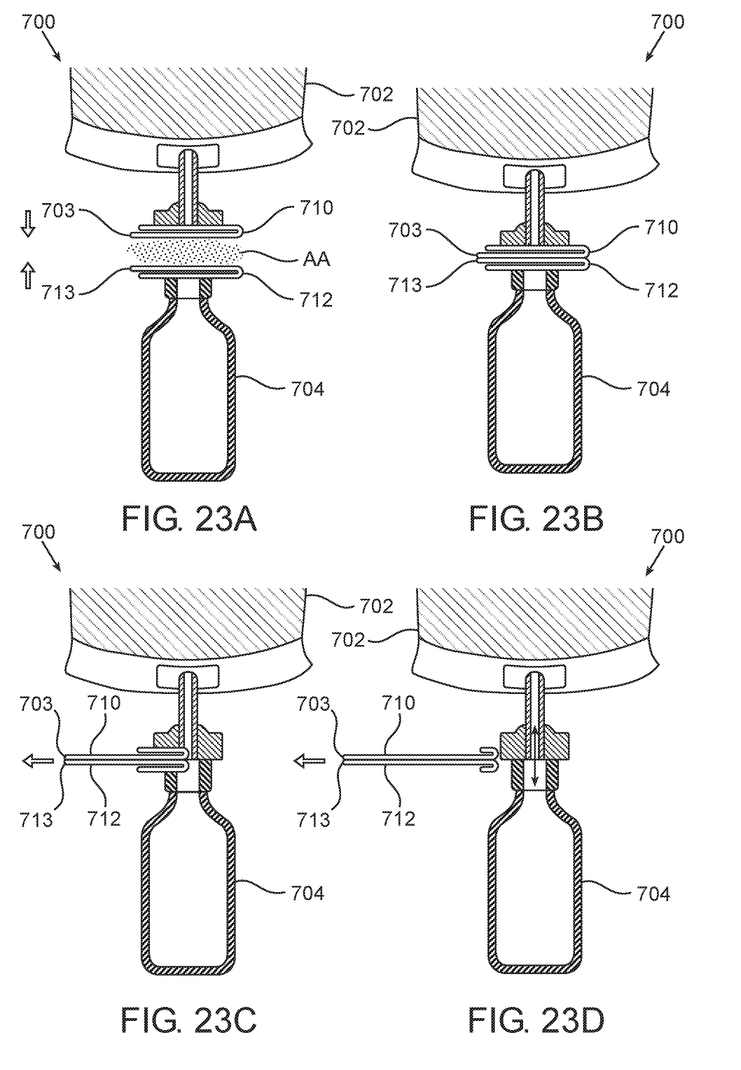

Reference is now made to FIGS. 23A-23D which demonstrate yet a further exemplary decontamination system 700 which includes a first connection interface 710 attached to a first vessel 702 and a second connection interface 712 attached to a second vessel 704. The connection interfaces 710 and 712 are shown when in a folded position. It is to be noted that either or both connection interfaces 710 and 712 can be in a folded or unfolded position (not shown). FIG. 23A shows system 700 prior to engagement between the vessels 702 and 704. FIG. 23B depicts system 700 wherein the interfaces 710 and 712 when in a folded position attached to each other and a hermetic connection formed between vessels 702 and 704. FIG. 23C shows the connection interfaces 710 and 712 while externally displacing, by peeling off motion, from the vessels 702 and 704. The vessels 702 and 704 are resealed at about the time or prior to the external displacement. The vessels are hermetically engaged during peeling of the interfaces 710 and 712. FIG. 23D shows the connection interfaces 710 and 712 following the external displacement. A hermetic seal between vessels 702 and 704 is maintained by pressure of engagement between both vessels 702 and 704 provided by the engagement mechanism (not shown) on vessel 702 and/or on vessel 704. Resealing of the aperture or valve (not shown) through which connection interfaces 710 and 712 pass through occurs at about the time or prior to external displacement of connection interfaces 710 and 712 via the peeling motion. Resealing of the aperture or valve is provided by the force of engagement between first vessel 702 and second vessel 704 provided by an engagement mechanism that connects vessel 702 to vessel 704. The engagement mechanism (not shown) may be a thread, a luer, a ratchet teeth mechanism, a slide-on mechanism, a snap-on mechanism, a clamp rail mechanism, an adhesive mechanism or combinations thereof. In one or more embodiments, the folded position of connection interfaces 710 and/or 712 provides two film layers for each connection interface. In an embodiment of the invention, the folded position may include protrusions 703 and 713 of one of the layers, allowing usage thereof as a tab that can be peeled out to allow the external displacement. It is to be noted that the peeling mechanism may be relevant to any of the herein disclosed connectors and/or connection interfaces. External displacement of the connection interfaces 710 and/or 712 via a peeling off motion may occur between teeth of a ratchet teeth engagement mechanism or between threads of a thread/luer engagement mechanism. The peeling off motion of the connection interfaces 710 and 712 may occur via a pulling motion.

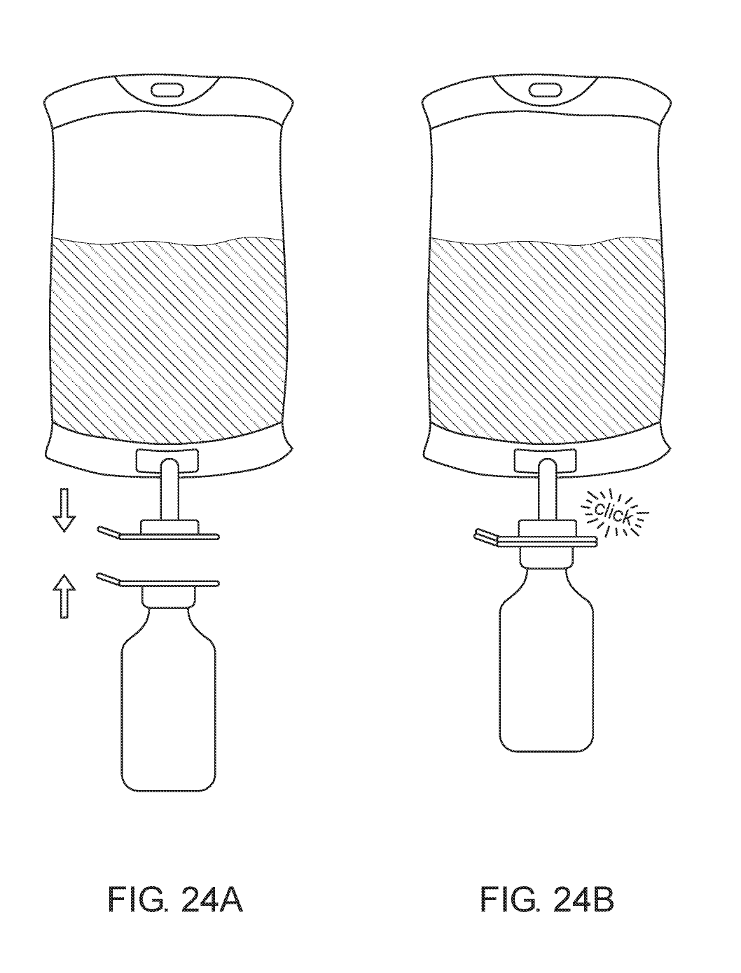

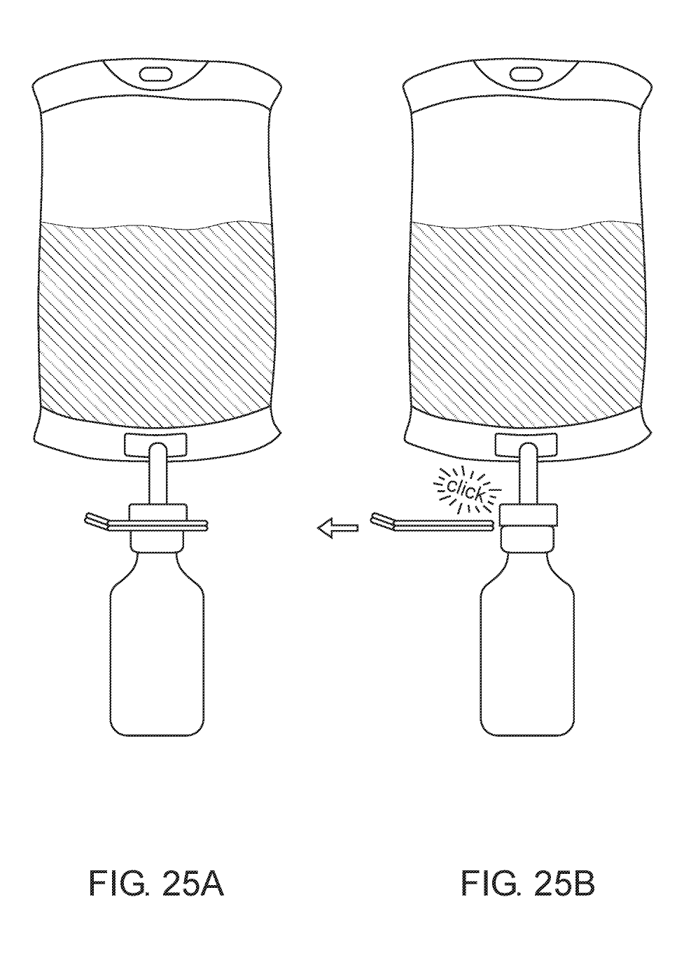

Reference is now made to FIGS. 24A-24B which schematic illustration of yet a further exemplary decontamination system, wherein a click sound is heard at about the time of engagement of the vessels. In an alternative embodiment, a click sound is heard at about the time of external displacement of the vessels (FIGS. 25A-25B). It is to be noted that the click sound may be relevant to any of the herein disclosed systems and connectors. In an embodiment of the invention, the click sound alerts the user that both vessels have established a hermetic engagement. In an embodiment of the invention, the click sound alerts that one or more valves have been opened. In embodiment of the invention, the click sound alerts that one or more connection interfaces has been externally displaced. In one or more embodiments, the click sound may be another sound. In one or more embodiments, the sound is audible to a user of the system.

In an aspect of the invention, the present invention provides a method of decontaminating a connection between two vessels, the method comprising the steps of: providing a first connection interface attached to a first vessel; providing a second connection interface attached to a second vessel, engaging the first vessel to the second vessel; and externally displacing the first and second connection interfaces from the engagement between the first vessel and the second vessel, while a hermetically sealed connection between the first and second vessels is maintained.

In yet a further aspect, the present invention provides a method of decontaminating a connection between two vessels, the method comprising the steps of: providing a first connection interface attached to a first vessel; providing a second vessel; engaging the first vessel to the second vessel; and externally displacing the first connection interface from the engagement between the first vessel and the second vessel, while a hermetically sealed connection between the first and second vessel is maintained.

In one or more embodiments, the step of externally displacing the first connection interface and the second connection interface occurs via a sliding motion.

In one or more embodiments, the step of externally displacing the first connection interface and the second connection interface occurs via sliding axially with respect to the first vessel and the second vessel, thereby removing contaminants located between the first and second interfaces.

In one or more embodiments, an airtight seal is formed between the first connection interface and the second connection interface prior to the external displacement of the first and second connection interfaces.

In one or more embodiments, the displacement of the first connection interface and the second connection interface is via a rail mechanism, such that the airtight sealed compartment between the first connection interface and the second connection interface slides off the rail.

In one or more embodiments, when the airtight seal between the first connection interface and the second connection interface is displaced, the connection between the first vessel and the second vessel is decontaminated.

In one or more embodiments, the airtight seal between the first connection interface and the second connection interfaces slides off the rail while maintaining the hermetically sealed connection between the first and second vessels.

In one or more embodiments, the step of externally displacing the first connection interface and the second connection interface occurs via peeling.

In one or more embodiments, the step of externally displacing the first connection interface and the second connection interface occurs via a hinge mechanism.

In one or more embodiments, the step of externally displacing the first connection interface and the second connection interface occurs at about the same time. In one or more embodiments, the step of externally displacing the first connection interface and the second connection interface occurs at the same time. In one or more embodiments, the step of externally displacing the first connection interface and the second connection interface occurs sequentially.

In one or more embodiments, the method further comprises connecting the first connection interface to the second connection interface via an engagement mechanism.

In one or more embodiments, connecting of the first connection interface to the second connection interface occurs prior to externally displacing the first and second connection interfaces from the engagement between the first vessel and the second vessel. In one or more embodiments, connecting of the first connection interface to the second connection interface occurs at about the time of external displacement of the first and second connection interfaces from the first and second vessels.

In one or more embodiments, the connecting of the first connection interface to the second connection interface is selected from a twisting of a thread onto a complementary thread, attaching ratchet teeth to a complementary retention member, and adhering a first connection interface to the second connection interface via an adhesive.

In one or more embodiments, the sealed aperture is a valve. In one or more embodiments, the valve is a one-way valve.

In one or more embodiments, the sealed aperture reseals at about the time of external displacement of the first and second connection interfaces.

In one or more embodiments, the sealed aperture reseals prior to detachment of the first and second connection interfaces from the first and second vessels.

In one or more embodiments, at least one of the first and second vessels is a container. In one or more embodiments, the container is a bag or a bottle. In one or more embodiments, at least one of the first and second vessels is a syringe. In one or more embodiments, the container has a flexible wall.

Each of the following terms: ‘includes’, ‘including’, ‘has’, ‘having’, ‘comprises’, and ‘comprising’, and, their linguistic, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably.

The term ‘about’, is some embodiments, refers to ±30% of the stated numerical value. In further embodiments, the term refers to ±20% of the stated numerical value. In yet further embodiments, the term refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A container housing a substance intended for intracorporal administration to a patient, the container comprising:
a rail mechanism, and
an externally displaceable connection interface,
wherein the rail mechanism is configured to provide for the external displacement of the connection interface.

2. The container of claim 1, wherein the container further comprises an engagement mechanism.

3. The container of claim 2, wherein the engagement mechanism is selected from a group consisting of a thread, a ratchet teeth mechanism, an adhesive, a snap-on mechanism and combinations thereof.

4. The container of claim 1, wherein the connection interface is disposed on the rail mechanism.

5. The container of claim 1, wherein the connection interface is disposed between rails of the rail mechanism.

6. The container of claim 1, wherein the connection interface is attached to the rail mechanism via a frangible seal, wherein the frangible seal is configured to break/rupture by a force of a user's hand upon displacement of the connection interface from the rail.

7. The container of claim 1, wherein the container has a flexible wall.

8. The container of claim 1, wherein the container has a rigid wall.

9. The container of claim 1, wherein the container is a bag.

10. The container of claim 1, wherein the container is a bottle.

11. The container of claim 1, wherein the container is a vial.

12. The container of claim 1, wherein the container is a syringe.

13. The container of claim 1, wherein the connection interface displaces between teeth of a ratchet teeth engagement mechanism.

14. The container of claim 1, wherein the connection interface displaces between threads of a thread engagement mechanism.

15. The container of claim 1, wherein the connection interface has an adhesive on a surface of the connection interface.

16. The container of claim 18, wherein the connection interface has at least one of a disinfecting agent and a sterilizing agent disposed on the surface of the connection interface.

17. A decontamination connector for the decontamination of a connection between vessels, comprising:
a port;
a connection interface configured to cover said port of said decontamination connector, wherein said connection interface is configured to displace externally from said port of said decontamination connector;
a sealed aperture configured to provide for the external displacement of said connection interface; and
an engagement mechanism, wherein the said engagement mechanism is configured to provide coupling between said port of the decontamination connector and a vessel, wherein following said coupling, said decontamination connector and said vessel form a hermetically coupled connection, while said connection interface is externally displaced from said port of said decontamination connector through the sealed aperture while the hermetically coupled connection between said decontamination connector and said vessel is maintained.

18. The decontamination connector of claim 17, wherein resealing of the sealed aperture occurs at about the time of external displacement of the connection interface.

19. The decontamination connector of claim 17, wherein resealing of the sealed aperture occurs after external displacement of the connection interface.

20. The decontamination connector of claim 17, wherein the sealed aperture provides for the external displacement of at least two connection interfaces.
